(19) United States
(12) Patent Application Publication
(10) Pub. No.: US 2006/0251625 A1
(43) Pub. Date: Nov. 9, 2006

(54) MCV MC160 compositions and methods

(76) Inventors: Joanna Shisler, Urbana, IL (US); Daniel Brian Nichols, Champaign, IL (US)

Correspondence Address: GREENLEE WINNER AND SULLIVAN P C, 4875 PEARL EAST CIRCLE, SUITE 200, BOULDER, CO, 80301, US

(21) Appl. No.: 11/429,671

(22) Filed: May 5, 2006

Related U.S. Application Data
(60) Provisional application No. 60/677,907, filed on May 5, 2005.

(51) Int. Cl. C12N 15/86; A61K 48/00

(52) U.S. Cl. 424/93.2; 435/456

(57) ABSTRACT

Provided are therapeutic compositions having anti-inflammatory activity in a human or an animal, said compositions comprising a Molluscum contagiosum virus MC160 protein or an inflammation-inhibiting peptide or polypeptide derived in amino acid sequence therefrom. Optionally, these compositions can further comprise a cell penetration peptide, such that the MC160 protein, peptide or polypeptide is transported into cells of the human or animal, with the result that the inflammatory response is reduced at a site negatively impacted by inflammation. Methods for reducing the inflammatory response in a human or animal in need of same are provided, and these methods include the step of applying or administering an effective amount of the relevant composition.

### CROSS-REFERENCE TO RELATED APPLICATIONS

This application claims benefit of U.S. Provisional Application 60/677,907, filed May 5, 2005.

### STATEMENT REGARDING FEDERALLY SPONSORED RESEARCH OR DEVELOPMENT

This invention was made with government support under Grant No. Al 05530 awarded by the National Institutes of Health. The government has certain rights to the invention.

### REFERENCE TO SEQUENCE LISTING, A TABLE, OR A COMPUTER PROGRAM LISTING COMPACT DISK APPENDIX

SEQ ID NO:1, MCV MC160 protein sequence.

SEQ ID NO:2, cell penetration peptide corresponding to amino acids 47-57 of Human Immunodeficiency Virus Tat protein.

SEQ ID NO:3, cell penetration peptide corresponding to amino acids 48-60 of Human Immunodeficiency Virus Tat protein.

SEQ ID NO:4, cell penetration peptide corresponding to amino acids 43-58 of Drosophila Antennapedia protein.

SEQ ID NO:5, cell penetration peptide corresponding to amino acids 35-47 of human Clock protein.

SEQ ID NO:6, cell penetration peptide corresponding to amino acids 831-845 of hPer1 protein.

SEQ ID NO:7, cell penetration peptide corresponding to amino acids of 789-806 of hPer2 protein.

SEQ ID NO:8, synthetic cell penetration peptide (Pep-1).

SEQ ID NO:9, synthetic peptide with amino acid sequence unique to MCV MC160.

SEQ ID NO:10, oligonucleotide useful as forward primer for human TNF (Tumor Necrosis Factor).

SEQ ID NO:11, oligonucleotide useful as reverse primer for human TNF.

SEQ ID NO:12, oligonucleotide useful as forward primer for human GAPDH.

SEQ ID NO:13, oligonucleotide useful as reverse primer for human GAPDH.

SEQ ID NO:14, oligonucleotide containing binding site(s) for the NF-κB transcription factor.

### BACKGROUND OF THE INVENTION

The field of this invention relates to the inflammatory process and methods for decreasing the inflammatory response. In particular, the inflammatory response is lessened by inhibiting NF-κB activation via the Molluscum contagiosum virus (MCV) MC160 protein or an effective polypeptide derived therefrom.

Molluscum contagiosum virus (MCV) is a dermatotropic poxvirus that causes skin neoplasms (12). Other than variola virus (the causative agent of smallpox), MCV is the only poxvirus that relies exclusively on humans as hosts. MCV infections are common and worldwide and causes a relatively benign infection in healthy individuals (16). These virus-filed lesions can persist for months without signs of inflammation, suggesting local immunosuppression. MCV lesions are much more extensive in AIDS patients, a fact which reflects the importance of the host immune response in controlling virus replication (28). No anti-viral drug treatment successfully eliminates MCV infections; therefore, physical methods such as curettage or cryotherapy are required for lesion removal.

MCV-infected skin lesions lack immune effector cells and molecules, suggesting that MCV infection dampens localized inflammatory responses (2, 13, 27). Analysis of the nucleotide sequence of the entire MCV genome revealed the presence of multiple genes whose products possess anti-inflammatory activities (21, 23, 24). The MCV MC160 gene product exhibits significant amino acid sequence homology to cellular and viral proteins that bind to IKK (I Kappa Kinase) and thus regulate NF-κB activity (5, 15, 18, 25).

The NF-κB transcription factor family induces the expression of proinflammatory cytokines (11). NF-κB family members form catalytically active hetero- or homodimers, with the p65:p50 dimer being the most abundant and best-characterized complex (14) (FIG. 1). In resting cells, NF-κB is inhibited by a family of inhibitory proteins (IκBs) that bind to NF-κB. IκBα is the most abundant and well characterized of these inhibitors, binding with the highest frequency to the p65 subunit of the p65:p50 heterodimer (FIG. 1). As a result of this interaction, NF-κB is unable to bind to its conserved DNA consensus sequence (5′-GGGRNNYYC-3′) in cellular gene promoters (11, 14).

The TNF-RI is the TNF-α cellular receptor that mediates NF-κB activation or apoptosis (FIG. 1). For NF-κB activation, the cellular TNF Receptor Associated Death Domain TRADD, Receptor Interacting Protein RIP, and TNF Receptor Associated Factor 2 TRAF2 proteins form a receptor-associated signalsome complex (FIG. 1). Inactive IKK migrates to this complex, where it is activated (FIG. 1). Activated IKK kinase phosphorylates IκBα at serine residues 32 and 36 (3, 4, 8, 9, 19, 26). Next, IκBα is polyubiquitinated at lysines 21 and 22. As a result, IκBα is released from the NF-κB dimer and migrates to the 26S proteosome where it is degraded (1, 7, 8, 22). The free NF-κB translocates to the nucleus; then it binds to its nucleotide consensus sequence and activates transcription.

For apoptosis, TRADD, FADD and procaspase-8 migrate to the receptor (FIG. 1), where procaspase-8 is proteolytically cleaved to its active form. Caspase-8 cleaves and activates downstream effector caspases, resulting in DNA fragmentation and subsequent cell death.

To circumvent inflammation and possibly other host immune responses, some pathogens such as poxviruses express proteins designed to dampen these defense mechanisms by preventing either an activated NF-κB moiety from functioning in the nucleus or a step in the activation process (30). For instance, the Leporipoxvirus, myxoma virus, utilizes the former strategy by expressing the M150R protein that presumably associates with NF-κB at a nuclear location (31). In contrast, the Orthopoxvirus, vaccinia virus, expresses the N1L, A46R, and A52R proteins to inhibit Toll-like receptor mediated NF-κB activation (32, 32). The Molluscipoxvirus, Molluscum contagiosum virus (MCV) appears to be in the later category in that its MC159 protein blocks death-receptor induced activation of NF-κB, by acting on cytoplasmic events (5).

Inflammatory diseases are relatively common in the population, ranging from hypersensitive reactions and dermatologic conditions, rheumatoid arthritis and life-threatening diseases such as Crohn's disease and inflammatory bowel disease. There is significant pain, discomfort and illness associated with these disorders, as well substantial economic cost.

Because inflammatory disorders have a high economic cost and a high human cost in terms of patient discomfort and impaired function, there is a long felt need in the art for compounds which reduce inflammation, the attendant discomfort, limitation of mobility and the like.

### BRIEF SUMMARY OF THE INVENTION

The present invention provides methods for inhibiting Tumor Necrosis Factor-mediated activation of the transcription factor NF-κB, by administering an effective amount of the Molluscum contagiosum virus MC160 protein or a peptide derived in sequence from the MC160 protein which is effective for inhibiting activation of the transcription factor NF-κB. A beneficial result of this inhibition is that inflammation resulting from NF-κB activation is less than when MC160 is not present. The amino acid sequence of MC160 is set forth in SEQ ID NO:1. A truncated MC160 protein comprising amino acids 1-170 or 82-371 or 169-371 of SEQ ID NO:1 is useful for inhibiting NF-κB activation. Another truncated MC160 proteins useful for inhibiting NF-κB activation comprises amino acids 82-220 of SEQ ID NO:1.

The present invention also encompasses methods for reducing inflammation in a patient in need of such treatment. Inflammation is reduced by the administration of a composition comprising a pharmaceutically acceptable carrier and MC160 protein or an inflammation-reducing derivative thereof in an amount effective to reduce inflammation. Where the inflammation to be treated is on the skin, a topical composition comprising the MC160 is applied to the inflamed area of the skin.

Where the inflammation is in one or more joints, for example, in joints affected by rheumatoid arthritis, a composition containing MC160 or an effective peptide derivative thereof can be injected intravenously or into the affected joint in an amount effective for reducing inflammation and the discomfort to the patient.

Where the patient suffers from an inflammatory disorder such as Crohn's disease, a composition suitable for oral administration or parenteral, for example, intravenous, administration comprising the MC160 protein or an effective peptide derivative thereof in an amount effective for reducing inflammation in the small and/or large intestine of the patient.

Other diseases which can be ameliorated by the administration of the MC160 protein or an inflammation-reducing derivative thereof include, without limitation, juvenile rheumatoid arthritis, psoriatic arthritis, ankylosing spondylitis, ureitis, psoriasis and sarcoidosis.

### BRIEF DESCRIPTION OF THE DRAWINGS

FIG. 1 illustrates the TNF-R1-mediated NF-κB activation and apoptosis pathways, as discussed herein. Activated IKK is denoted by an asterisk (*). The MC160 protein was originally thought to bind to IKK to inhibit its migration to the TNF-R1. Indeed, MC160 inhibits IKK kinase activity.

FIG. 2 shows that the MC160 protein inhibits TNF-induced NF-κB activation. 293T cells were transfected with 1 μg pCI or MC160/pCI. 24 hours later, cells were incubated in media absent for or containing TNF for the indicated times. Nuclear-extracted proteins were analyzed for the presence of active NF-kB by a gel mobility shift assay. Briefly, nuclear extracted proteins are incubated with radiolabeled oligonucleotides containing a consensus NF-kB DNA binding sequence. These reactions are analyzed by gel electrophoresis under non-denaturing conditions. Where NF-kB is present in nuclear extracts, it binds to the oligonucleotide and retards the mobility of the oligonucleotide in the gel. This is detected by autoradiography. Anti-p65 or anti-50 antisera recognizing the NF-κB p65:p50 heterodimer was present in some reactions (denoted as “p65” or “p50”). “NF” and “AP” refer to reactions in which excess amounts of non-radiolabeled oligonucleotides containing NF-κB binding sites (NF) or no NF-κB binding sites (AP) were added to reactions. An asterisk (*) indicates the NF-κB-oligonucleotide complex, and a plus (+) indicates a super-shifted complex.

FIGS. 3A-3C show the effect of MC160 expression on TNF-α-induced expression of an NF-κB transcriptionally regulated luciferase gene. In FIGS. 3A and 3B, 293T cells were co-transfected with 450 ng of pNFκ-Bluc, 50 ng pRL-null, and 500 ng of either pCI or MC160/pCI. After 24 h: FIG. 3A: Cells were incubated in medium lacking or containing TNF (0-20 ng/ml) for 4 h and then harvested. Their lysates were analyzed for luciferase activity. FIG. 3B: Cells were incubated with medium lacking or containing 10 ng TNF/ml and cells were harvested either 4, 6, 8 or 12 h later. Then, their lysates were analyzed for luciferase activities. FIG. 3C: Cells were transfected with reporter plasmids and the indicated amounts of either pCI or a combination of pCI and MC160/pCI. Following a 24 h incubation, cells were incubated in medium lacking or containing TNF (10 ng/ml) and harvested 4 h later. Lysates were analyzed for luciferase activities. An asterisk (*) indicates statistically significant inhibition (p<0.05). For all experiments, 10 μg of each cellular lysate was screened for MC160 expression by immunoblotting (“IB”) with anti-MC160 protein antisera, as illustrated by the immunoblot inset above each graph.

FIG. 4 shows the effect of MC160 expression on TNF-α-induced NF-κB nuclear translocation. 293T cells were transfected with either 1 μg pCI or MC160/pCI. 24 h later cells were incubated in medium lacking (−−) or containing TNF (10 ng/ml) for the times indicated. Cells were harvested, lysed, and nuclei were separated from cell lysates. Two μg of nuclear protein from each sample were incubated with radiolabeled oligonucleotides containing consensus NF-κB binding sites. Nuclear extracts from pCI-transfected cells treated with TNF for 45 min were also incubated with either non-radiolabeled oligonucleotides (NF), oligonucleotides devoid of an NF-κB consensus sequence (AP), anti-p50 (p50) or anti-p65 (p65) antisera. The lane labeled “no lys” indicates that no nuclear-extracted proteins were present in the reaction. Reactions were resolved by 6% PAGE under non-denaturing conditions. A mobility-retarded complex containing NF-κB is indicated by a plus symbol (+) while super-shifted NF-κB-containing complexes are denoted by a double plus symbol (++). A nonspecific band and the band composed of unbound oligonucleotides are denoted by a single (*) or double asterisk (**), respectively.

FIGS. 5A-5D show the effect of MC160 expression on either RIP-, TRAF2-, NIK-, or MyD88-induced NF-κB activation. 293T cells were co-transfected with pNF-κBluc (450 ng), pRL-null (50 ng), either pCI (500 ng) or MC160/pCI (500 ng), and either (FIG. 5A) pHA-RIP (50-500 ng) or (FIG. 5B) pHA-TRAF2 (500-2000 ng) or (FIG. 5C) pNIK (10-50 ng) or (FIG. 5D) pFlagMyD88 (250 ng). A separate set of cells were transfected with 500 ng MC160/pCI and 500 ng pIKK2DN in FIG. 5D only. At 24 hours post-transfection, cells were harvested and lysates were analyzed for luciferase activities. An asterisk (*) denotes statistically significant inhibition (p<0.05).

FIGS. 6A-6B show the effect of MC160 expression on IKK activation. FIG. 6A: 293T cells were co-transfected with 450 ng pNF-κBluc, 50 ng pRL-null, 500 ng of either pCI or MC160/pCI, and 500 ng of either pFLAG-IKK1 or pHA-IKK2. 24 h later, cells were harvested and lysates were assayed for luciferase activities. An asterisk (*) denotes statistically significant inhibition (p<0.05). FIG. 6B: 293T cells were co-transfected with 250 ng pFLAG-IKK1, 250 ng pHA-IKK2 and either 1.5 μg of pCI or MC160/pCI. 24 h later, cells were harvested, lysed and incubated with anti-IKK1 antibodies and Protein A-Sepharose beads. Immunoprecipitated samples were incubated with recombinant IκBα and (α-³²P) ATP in a kinase reaction buffer. Proteins were separated by 12% SDS-PAGE and transferred to PVDF membranes. The membranes were first exposed to phosphorimaging plates (upper panel), and then subsequently probed for IκBα by immunoblotting (lower panel), using an alkaline phosphatase-conjugated secondary antibody.

FIGS. 7A-7C show the effect of MC160 expression on IKK subunit interactions. FIG. 7A: 293T cells were co-transfected with 500 μg pFLAG-IKK1, 500 μg pHA-IKK2 and either 1 μg pCI or MC160/pCI. At 24 h post-transfection, cells were lysed and incubated with anti-HA antibody conjugated to protein A-Sepharose beads. Immunoprecipitated samples (“IP”) were separated by 12% SDS-PAGE and proteins were transferred to a PVDF membrane. Immunoblots (IB) were probed with the indicated antisera. FIG. 7B: 293 T cells were transfected with 1 μg of either pFlag-IKK1 or MC160/pCI. At 24 h post-transfection, cells were lysed and incubated with either anti-IKK1 antibody (IP) or with antisera non-specific for IKK1 (“C”) conjugated to protein A-Sepharose beads. Immunoprecipitated samples (“IP”) and control samples (“C”) were separated by 12% SDS-PAGE and proteins were transferred to a PVDF membrane. Immunoblots (IB) were probed with the indicated antisera. FIG. 7C: 293 T cells were transfected with 1 μg of either pCI or MC160/pCI. At 24 h post-transfection, cells were lysed and incubated with anti-IKK-γ conjugated to protein A-Sepharose beads. Immunoprecipitated samples were separated by 12% SDS-PAGE and proteins were transferred to a PVDF membrane. Immunoblots (IB) were probed with the indicated antisera. For all experiments, pre-immunoprecipitated lysates were also analyzed by immunoblotting for MC160 protein expression.

FIG. 8 shows the effect of MC160 expression on IKK1-IKK2 interactions in the presence or absence of TNF-α-Subconfluent 293T cells were co-transfected with 500 μg pFLAG-IKK1, 500 μg pHA-IKK2 and 1 μg of either pCI or MC160/pCI. At 24 h post-transfection, fresh media was added either lacking (−) or containing (+) 10 ng/ml TNF-[ ]Following a 15 minute incubation, cells were lysed, and lysates were incubated with Protein A-Sepharose beads and either anti-HA or mouse IgG antibody. Immunoprecipitated samples (“IP”) were separated by 12% SDS-PAGE and proteins were transferred to a PVDF membrane. Immunoblots (IB) were probed with the indicated antisera. As a control, pre-immunoprecipitated lysates were also analyzed by immunoblotting for MC160 protein expression.

FIG. 9 illustrates the levels of phosphorylated and unphosphorylated IKK1 and IKK2 proteins in the presence of MC160. Subconfluent 293T cells were transfected with either 250 ng of pFlag-IKK1 and 250 ng of pHA-IKK2, or 500 ng of pFlag-IKK1, or 500 ng of pHA-IKK2. In addition, cells were simultaneously co-transfected with 500 ng of either pCI, MC160/pCI or MC159/pCI. At 24 hours post transfection, cells were lysed and 30 μg of cytoplasmic extracts were separated by 12% SDS-PAGE. Proteins were electrophoretically transferred to PVDF membrane, and immunoblots (IB) were probed with the indicated antisera.

FIGS. 10A-10B illustrate the effect of mutant MC160 proteins on TNF-induced NF-κB activation. FIG. 10A: Wild type and mutant MC160 proteins used for luciferase assays. FIG. 10B: 293T cells were transfected with luciferase and pCI-based plasmids. 24 hours later, cells were treated with TNF for 4 hours. Lysates were analyzed for firefly and sea pansy luciferase activities.

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

A cell penetration peptide is one which has a membrane permeability and carrier function for intracellular protein delivery. Typically, such a protein has an overall net positive charge, and is likely to be rich in arginine and/or lysine. A cell penetration peptide useful in the present invention may include D-amino acids instead of or in addition to L-amino acids. Specific examples of cell penetration peptides include, but are not limited to, those corresponding in amino acid sequence to amino acids 47 to 57 (YGRKKRRQRRR, SEQ ID NO:2) and amino acids 48-60 (RKKRRQRRRAHQ, SEQ ID NO:3) of the HIV type 1 Tat protein (Vives et al. 1997. J. Biol. Chem. 272(25):16010-16017), amino acids 43-58 of the Antennapedia protein (RQRIKIWFQNRRMKWKK, SEQ ID NO:4) from Drosophila (Christiaens et al. 2004. Eur. J. Biochem. 271:1187-1197; Derossi et al. 1996. J. Biol. Chem. 271(30):18188-18193), amino acids 35 to 47 of the human Clock protein (RVSRNKSEKKR, SEQ ID NO:5), amino acids 831-845 of hPerI (RRHHCRSKAKRSRHH, SEQ ID NO:6) and amino acids 789-806 of hPer2 (KKTGKNRKLKSKRVKPRD, SEQ ID NO:7) (Peng et al. 2004. Acta Biochim. Biophys. Sinica 36:629-636), as well peptides derived in sequence from HIC Rev, flock house virus coat proteins, DNA binding segments of leucine zipper proteins including the cancer-related proteins c-Fos and c-Jun, and the yeast transcription factor GCN4. US Patent Publication No. 2003/0185862 describes fusion proteins containing the sequence RKKRRQRRR (amino acids 3 to 11 of SEQ ID NO:2, derived from the HIV Tat protein) which are efficiently transported into cells via the Tat-derived sequence. An additional cell penetration peptide (KETWWETWWTEWSQPKKKRKV, SEQ ID NO:8) is described herein and in Morris et al. 2001. Nat. Biotechnol. 19:1173-1176. See also Schwarze et al. 1999. Science 285:1569-1572; Futaki et al. 2001. J. Biol. Chem. 276:5836-5840; Schwarze et al. 2000. Trends Pharmacol. Sci. 21:45-48, for a discussion of peptide-mediated intracellular transport. In the practice of the present invention, the cell penetration peptide can be covalently attached to the inflammation-inhibiting protein, e.g., it can be expressed as part of a fusion protein expressed via recombinant DNA technology. Alternatively, the cell penetration peptide can be mixed with the inflammation-inhibiting protein in the therapeutic composition.

To show that MC160 inhibits TNF-induced NF-κB activation and binds to IKK, an electromobility shift assay (EMSA) was employed to detect NF-κB activation. Using this technology TNF-induced NF-κB activation was demonstrated in pCI- or MC160/pCI-transfected cells using a gel mobility shift assay.

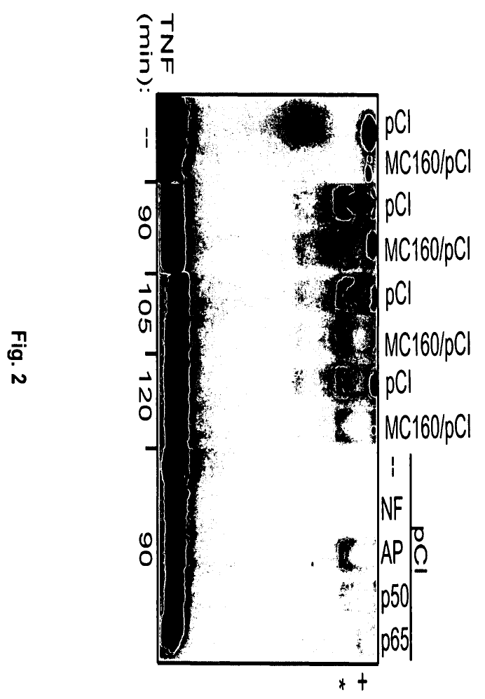

The MC160/pCI construct, antisera recognizing MC160, and methods used to transiently transfect plasmids into 293T cells, are described in Shisler and Moss (2001). The presence of a mobility-retarded band in TNF-treated, vector-transfected cells showed that TNF induced NF-κB activation (FIG. 2). In contrast, MC160 expression prevented TNF-induced NF-κB activation, as was evidenced by less intense bands (FIG. 2).

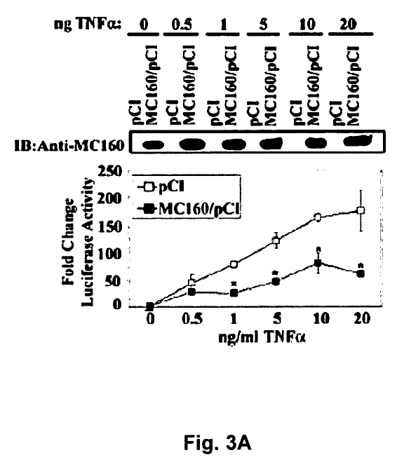

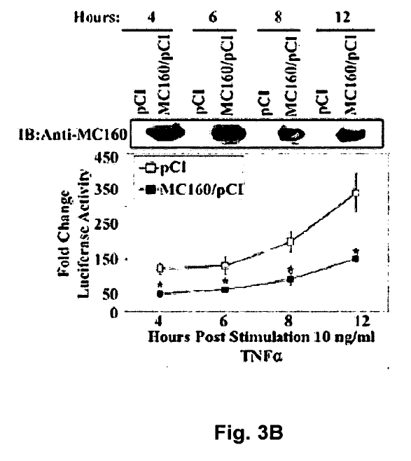

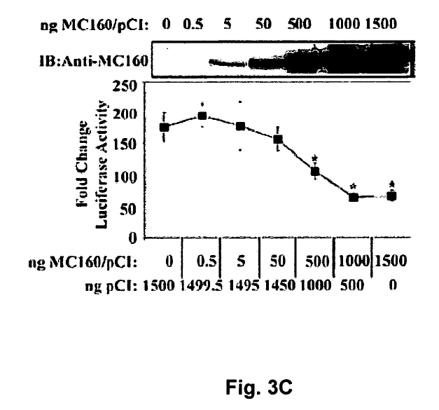

MC160 is partially homologous to other viral and cellular proteins known to bind to IKK (15, 23). We confirmed that MC160 interacts with IKK to inhibit NF-κB activation. To detect MC160-IKK interactions, MC160 and the IKK2 subunit were over-expressed in 293T cells. IKK2 (and therefore the entire IKK complex) was immunoprecipitated from cell lysates, and the presence of MC160 in immunoprecipitated samples was detected by immunoblotting (see Shisler and Moss (2001) for a detailed co-immunoprecipitation protocol). As shown in FIG. 3, MC160 was present only in immunoprecipitates from cells over-expressing both MC160 and either IKK1 or IKK2, indicating that MC160 interacted either directly or indirectly with IKK.

Binding of MC160 to cellular proteins does not necessarily indicate biological relevance. For example, MC160 associates with FADD or procaspase-8, but does not inhibit apoptosis (25). Therefore, we demonstrated that MC160 association with IKK is correlated with an inhibitory function.

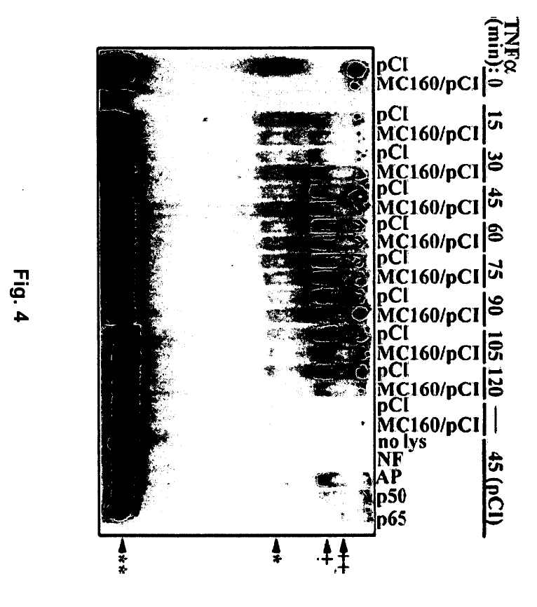

We constructed truncated MC160 ORFs that expressed either the MC160 DED 1 or DED 2 regions (see below) or the C-terminal region (FIG. 4A) as in-frame hemagglutinin tag fusions, and we employed a luciferase-based reporter assay to measure TNF-induced NF-κB activation. For these assays an ectopically-introduced luciferase gene was expressed under the regulatory control of an NF-κB-specific promoter such that the luciferase enzyme was synthesized only when NF-κB is active. See Shisler and Jin for a detailed luciferase-reporter assay protocol.

293T cells were co-transfected with plasmids containing luciferase genes and either wild type or mutant MC160 plasmids. 24 hours later, cells were incubated with TNF and harvested and luciferase activities in lysates were measured. All proteins possessed an N-terminal hemagglutinin (HA) epitope tag to allow for verification of mutant protein expression by immunoblotting.

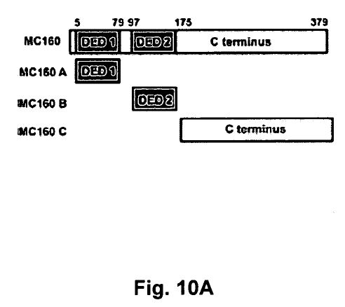

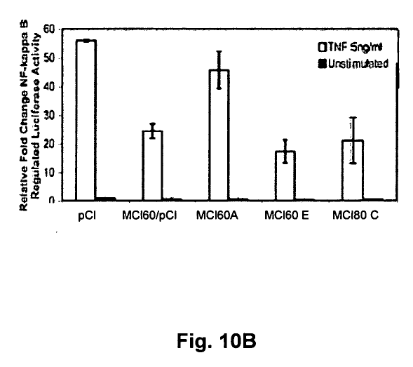

Wild type MC160 expression decreased luciferase activity as compared to vector-transfected cells, confirming the EMSA results. Expression of MC160 DED 2 (a 138 amino acid product), but not DED 1, also inhibited luciferase activity (FIG. 10B). Hu et al. (1997) reported that the DED 1 of MC160 corresponds to amino acids 7-71, and the DED 2 of MC160 corresponds to amino acids 92-164 of MC160. The full length MC160 protein sequence is given in Table 1 and SEQ ID NO:1. The (truncated) recombinant DED 1 and DED 2 polypeptides described herein correspond to amino acids 1 to 74 and 82 to 220, respectively, of the full length protein. See FIG. 10A for a schematic display.

Because death effector domains (DED) are motifs present in other cellular and viral proteins that are important for protein-protein interactions, the working hypothesis is that the MC160 DED 2 binds to IKK to prevent IKK activation. Surprisingly, the MC160 C-terminal region, corresponding to amino acids 169 to 371 of the full length MC160 protein, also inhibited luciferase activity (FIG. 10B). This region shares no homology with other known proteins, and it is not known whether this region binds to IKK or utilizes a different mechanism to inhibit NF-κB.

To test whether MC160-IKK interactions are required for the inhibitory function, the mutant MC160 A, MC160 B and MC160 C proteins were assayed for their abilities to associate with IKK by using the co-immunoprecipitation assays described above. The co-immunoprecipitation results are compared with those from the luciferase assays (FIG. 4B).

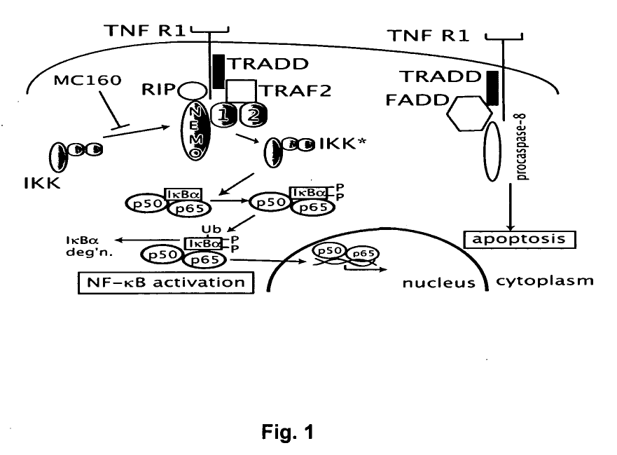

If there had been a 100% correlation between IKK binding and inhibitory function, then we would have concluded that IKK-MC160 interactions are necessary for inhibitory function. If there were no correlation between binding and inhibitory function, then we would have concluded that MC160 has more than one mechanism for inhibiting NF-κB. In that case, it is necessary to identify other functions including, but not limited to, IκBα phosphorylation and ubiquitination and migration of IKK, RIP and TRAF2 to the TNF-RI (FIG. 1). The data indicated there was not a 100% correlation between IKK binding and protection. Without wishing to be bound by any particular theory, we have concluded that MC160-IKK interactions are not the only mechanism used by MC160 to inhibit NF-kappa B activation.

IKK2 phosphorylation is required for TNF-induced NF-κB activation. Without wishing to be bound by any particular theory, the present inventors believe that MC160, MC160 B and MC160 C polypeptides each inhibits IKK2 activity. MC160 or IKK subunits (expressed by either an in vitro transcription-translation reaction or over-expression in E. coli) are incubated together in a cell-free system, and interactions are detected by co-immunoprecipitation assays.

Assaying the truncated MC160 A, MC160 B and MC160 C truncated proteins for IKK interacting function also provides useful information about the molecular mechanism of MC160. For example, if each mutant binds to a different IKK subunit, then MC160 possesses multiple mechanisms to prevent IKK activity. Alternatively, unique interactions of a particular MC160 region with one IKK subunit demonstrate the specific mechanism the MC160 protein utilizes. This information is useful in the design of MC160-derived small molecules to be used as anti-inflammatory agents.

During TNF-RI-mediated NF-κB activation, IKK migrates to the TRADD-RIP-TRAF2 signalsome (FIG. 1). Because MC160 interacts with IKK (FIG. 3), but not with RIP or TRAF2 (data not shown), one concludes that MC160 binds to IKK in the cytoplasm and prevents IKK migration to the signalsome. This is confirmed by using a TNF-RI co-immunoprecipitation protocol, similar to the one described for studying MC159 and MC160 migration to the Fas receptor (25).

Receptor co-immunoprecipitated samples are probed for MC160 and IKK by immunoblotting. If neither MC160 nor IKK is present in the TNF-RI signalsome, then the hypothesis is correct. If MC160 and IKK co-immunoprecipitate with the TNF-RI, then one concludes that that MC160 prevents IKK release from the signalsome, to inhibit IKK activation. If the latter is the case, then MC160 specifically inhibits only TNF-R1-mediated NF-κB activation, but does not inhibit other receptor-mediated signal transduction. If experiments prove this to be true, then MC160 therapeutic use is limited to that for TNF-mediated inflammatory diseases (such as rheumatoid arthritis) instead of for use as a broad spectrum anti-inflammatory. We have also found that MC160 inhibits MyD88- and NIK-induced NF-κB activation. Thus, the present inventors conclude that MC160 can act as a global inhibitor of classical NF-κB activation pathways.

The cellular mechanism that determines whether TRADD-RIP-TRAF2 molecules bind to the TNF-RI to induce NF-κB activation or TRADD-FADD-procaspase-8 molecules bind to the TNF-RI to induce apoptosis (FIG. 1) is not clear. A recent report suggests that segregation of the TNF-RI to lipid raft or non-raft regions of the plasma membrane determines whether the TNR-RI mediates apoptosis or NF-κB activation (17).

The MCV MC159 protein is homologous to MC160. MC159 is a 25-kDa protein that consists of two DEDs (25). MC159 inhibits TNF-induced apoptosis (FIG. 1) and also inhibits TNF-induced NF-κB activation. The MC160 protein does not protect against TNF-induced apoptosis (25), but it does inhibit TNF-RI-mediated NF-κB activation (5).

TNF-induced accessory molecule migration to the TNF-RI in MC160- or MC159-expressing cells is measured using the receptor co-immunoprecipitation assays described by Shisler and Moss (25). Thus, we assayed for the presence of TRAF2, RIP, FADD, procaspase-8 and MC159 or MC160. We also assayed for the presence or absence of TNF-RI in lipid and non-lipid rich plasma membrane regions in cells ectopically expressing MC159 or MC160. These assays determine whether the MC159 or MC160 proteins are signalsome components, whether MC159 or MC160 alters migration of cellular signalsome molecules to the TNF-RI and whether MC159 or MC160 alters TNF-RI localization to lipid raft or non-raft regions as a mechanism for inhibiting TNF-RI-mediated signaling. More importantly, in comparing the results of these receptor co-immunoprecipitation assays and lipid raft assays to the ability of MC159 or MC160 to inhibit apoptosis or NF-κB activation, we clarify how the formation of the apoptosis signalsome versus the NF-κB signalsome is regulated and define the cellular events necessary for either process.

NF-κB also activates anti-apoptosis molecules (i.e.; c-FLIP, cIAP-1, cIAP-2) (6). Healthy cells, in response to TNF, prevent TNF-induced apoptosis by expressing these NF-κB-regulated anti-apoptosis molecules. Because MC160 is expressed and inhibits NF-κB during MCV infection, MCV-infected host cells are sensitized to TNF-induced apoptosis, resulting in rapid clearance of MCV lesions. However, MCV-infected cells survive for lengthy periods of time, suggesting that the MC160 molecule either selectively inhibit NF-κB-mediated transcription or inhibit only a certain subset of NF-κB complexes.

To test whether MC160 inhibits the transcription of some or all TNF-induced genes, we detect the mRNA of anti-apoptosis and proinflammatory genes in MC160-expressing cells, by using reverse transcriptase PCR, northern blotting or ribonuclease protection assays. If MC160 does inhibit transcription of all genes tested, then we conclude that expression of the NF-κB-controlled survival molecules are not required in order to prevent apoptosis. More interesting results would be the selective inhibition of gene expression by MC160, suggesting at least two distinctly regulated NF-κB pathways induced by TNF: one for inflammation and one for cell survival. Further characterization of these cellular mechanisms facilitates the development of drugs which specifically regulate each signal transduction pathway.

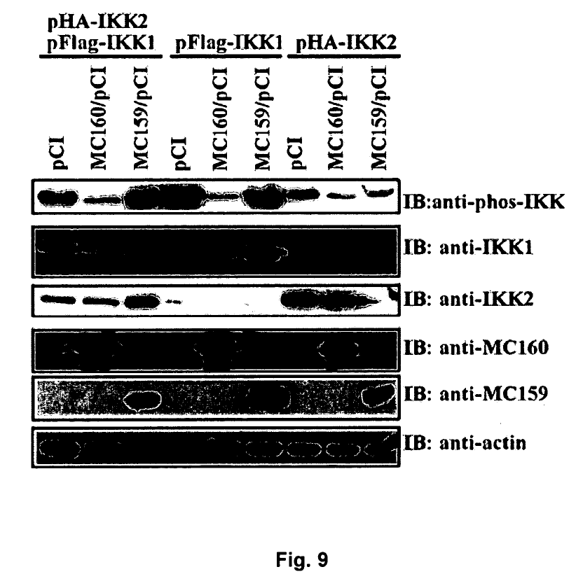

While IKK1 and IKK2 subunits are normally inactive in resting cells, ectopic over-expression of these proteins results in their phosphorylation and subsequent activation, an event detectable by probing immunoblotted lysates from pCI-transfected cells with antisera that simultaneously recognizes phosphorylated IKK1 and IKK2 (FIG. 9). In contrast, there was a decrease in phospho-IKK levels in MC160-expressing cells. This decrease was not due to over-expression of protein since expression of the MCV MC159 product did not significantly alter IKK phosphorylation levels when IKK1 and IKK2 were co-expressed. Further analysis of these lysates revealed that IKK1 levels, but not IKK2 or actin levels, were reduced in MC160-expressing cells as compared to either MC159/pCI- or pCI-transfected cells. Therefore, neither the transfection process nor MC160 expression globally decreased protein expression. Similar results were observed when only IKK1 was over-expressed: phospho-IKK1 and total IKK1 levels were decreased in MC160-expressing cells as compared to cells transfected with either pCI or MC159/pCI. Phosphorylation of IKK2 was also decreased in cells co-transfected with MC160/pCI and pHA-IKK2, indicating that MC160 inhibited activation of both kinase subunits. However, total IKK2 levels were similar in pCI- versus MC160/pCI-transfected cells, indicating that MC160 did not affect IKK2 protein levels. In MC159/pCI-transfected cells, IKK2 was present at levels similar to that seen in pCI-transfected cells, but phospho-IKK2 levels were decreased. Similar results were observed when cells were incubated in medium containing TNF-α, a cytokine known to induce IKK1 and IKK2 phosphorylation.

We have demonstrated that the MC160 protein inhibits the expression of an NF-κB-controlled reporter gene. MC160 protein inhibition of NF-κB activation was initially assessed by comparing the TNF-α-induced activities of an NF-κB-transcriptionally regulated firefly luciferase gene in MC160/pCI- and pCI-transfected cells (FIG. 3A-3C). In this experiment, TNF-α-mediated luciferase activity was reduced 1.5- to 3.1-fold in the presence of the MC160 protein (FIG. 3A). This reduction was statistically significant when determined after a four hour incubation in the presence of either 1 (p=0.0001), 5 (p=0.005), 10 (p=0.008) or 20 ng/ml (p=0.032) TNF-α. In comparison to exposure to 10 ng/ml TNF-α, luciferase activity was not greatly increased when pCI-transfected cells were incubated with either 20 (FIG. 3A) or 50 ng/ml TNF-α(data not shown). Thus, in the subsequent experiments, cells were incubated with 10 ng/ml TNF-α. It should also be noted that inclusion of TNF-α in the medium did not impact MC160 expression as similar amounts of this protein were detected in all of the MC160/pCI-transfected monolayers by immunoblotting (FIG. 3A). As vector-transfected cells were incubated for increasing times in the presence of 10 ng/ml TNF-α, luciferase activity levels increased (FIG. 3B). However, the MC160 protein significantly inhibited luciferase activity when the cells were examined at either 4 (p=0.005), 6 (p=0.037), 8 (p=0.011) or 12 (p=0.023) hours (FIG. 3B). Extended incubation of cells with TNF-αdid not drastically decrease MC160 expression since similar amounts of this protein were present at all times tested (FIG. 3B).

MC160-mediated inhibition of TNF-α-induced luciferase activity also correlated with the intracellular amount of this viral protein (FIG. 3C), further confirming the inhibitory function of MC160. Statistically significant reductions in luciferase activity were observed only when cells were transfected with 500 to 1500 ng MC160/pCI (p<0.02), conditions where MC160 expression was the greatest (FIG. 3C). In all experiments, MC160 expression did not induce luciferase activity in untreated cells, implying that MC160 did not activate NF-κB.

NF-κB transcriptional activation is reduced in MC160-expressing cells. NF-κB activation by TNF-α was also examined by using electromobility shift assays to measure binding of nuclear-located NF-κB to a radiolabeled oligonucleotide containing an NF-κB consensus sequence. As expected, nuclear extracts from unstimulated cell populations failed to alter oligonucleotide mobility, indicating that NF-κB was inactive (FIG. 4). In contrast, a unique mobility-shifted band (indicative of activated NF-κB) of similar intensities was observed when using nuclear extracts from pCI- and MC160/pCI-transfected cells stimulated with TNF-α for 15, 30 or 45 minutes (FIG. 4). However, the amount of the NF-κB-containing band (as denoted by “+”) was greatly reduced when extracts from MC160-expressing cells exposed to TNF-α for 60 to 120 minutes were used instead of ones from vector-transfected cells treated under the same conditions. Likewise, the addition of antibodies, specific for either the NF-κB p50 or p65 subunit, to extracts from TNF-α-treated, pCI-transfected cells decreased the signal intensity of the NF-κB-containing band (FIG. 4, lanes “p50” and “p65”) in comparison to samples lacking the antibodies. In general, the disappearance of the mobility-shifted band is evidence (albeit indirect) that anti-NF-κB antibodies specifically interacted with NF-κB. In a separate set of reactions, the addition of non-radiolabeled oligonucleotides containing NF-κB binding sites eliminated the NF-κB-containing band (FIG. 4, “NF”) while a similar quantity of nonspecific oligonucleotides had no effect (FIG. 4, “AP”). This specific competition demonstrated that NF-κB was indeed binding to NF-κB consensus sequences present in the radiolabeled oligonucleotides.

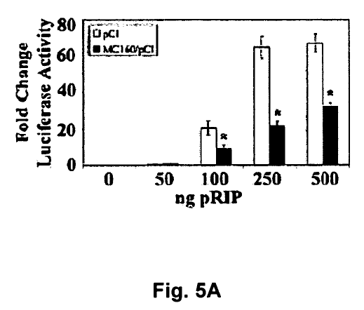

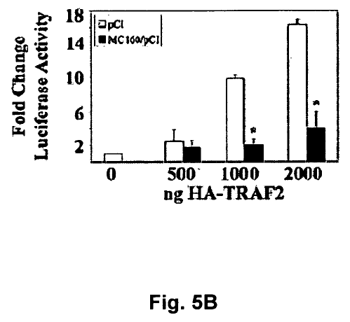

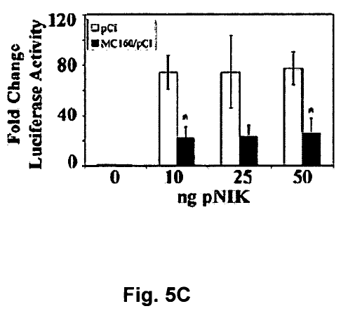

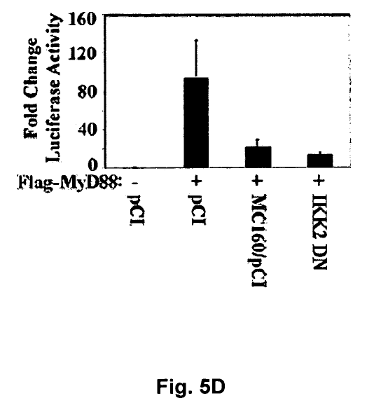

The MC160 protein inhibits either RIP, TRAF2-, NIK- or MyD88-induced NF-κB activation. After observing that NF-κB activation was inhibited in the presence of the MC160 protein, the next goal was to determine the step(s) of the TNF-RI-mediated NF-κB activation pathway that this viral protein blocked. In this regard, we studied the effect of MC160 expression on luciferase activity mediated by over-expression of either the RIP or TRAF2 accessory molecules. As shown in FIG. 5A, luciferase activity levels increased in cells transfected with 100-500 ng pRIP. In contrast, luciferase activity was 2- to 3-fold lower in cells co-expressing MC160 (FIG. 5A), and the decrease was statistically significant in cells transfected with MC160/pCI and either 100, 250 or 500 ng of pRIP (p<0.05). Increased RIP protein expression neither degraded MC160, nor prevented MC160 expression since the levels of this viral protein were similar in the appropriate samples (data not shown). TRAF2 over-expression also increased intracellular luciferase activity (FIG. 5B), although not to the same extent as RIP over-expression, as previously reported (39). Similar to above, MC160 expression reduced TRAF2-mediated luciferase activity, in this case 4- to 8-fold (FIG. 5B), with statistically significant inhibition occurring when cells were transfected with 1000 (p=0.0001) or 2000 (p=0.006) ng pHA-TRAF2. Immunoblotting of the respective lysates indicated the presence of the MC160 protein and TRAF2 in transfected cell lysates, demonstrating that the production of either protein was not impacted by transfection with large amounts of expression vectors (data not shown).

To determine whether the MC160 protein could affect other NF-κB activation pathways, an assessment of whether this viral protein inhibited either NIK (NF-κB-inducing kinase)- or MyD88-induced NF-κB activation was made. When cells were transfected with a plasmid capable of expressing NIK, luciferase activity was enhanced as compared to that in the non-transfected cells (FIG. 5C). This increase was presumably due to the ability of NIK to phosphorylate IKK1 (57), thus indirectly activating NF-κB. However, NIK-induced luciferase activity was reduced substantially when the MC160 and NIK proteins were co-expressed (FIG. 5C), indicating that MC160 partially prevented NIK-mediated NF-κB activation. The reduction in luciferase activity when cells were co-transfected with MC160/pCI and either 10 or 50 ng pNIK was statistically significant (p=0.008 and p=0.007, respectively). Likewise, over-expression of MyD88, a mediator of Toll-like receptor-induced NF-κB activation (58), resulted in a strong induction of luciferase activity (FIG. 5D). However, co-expression of MC160 decreased this artificially induced enhancement by 4.4-fold (FIG. 5D). In this case, the amount of MC160/pCI necessary to cause inhibition of MyD88-induced NF-κB activation did not appear to be abnormally great since transfection of cells with a similar quantity of pIKK2DN, which should presumably result in the production of a comparable protein, in this case a dominant defective IKK2, squelched luciferase activity to a similar degree (FIG. 5D).

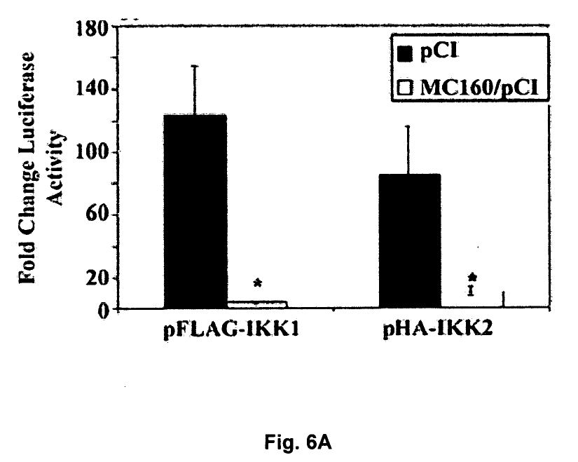

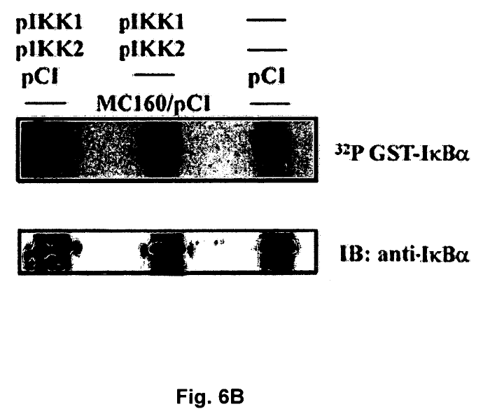

The MC160 Protein inhibits IKK kinase activity. Since the MC160 protein reduced signaling via molecules involved in TNF-α (namely RIP and TRAF2), and NIK- and MyD88-mediated activation of NF-κB, we predicted that this viral protein was inhibiting phosphorylation of cellular proteins by IKK, an event common to each of these pathways (40). In support of this hypothesis, we observed that MC160 expression was associated with statistically significant 35-fold reduction and 8-fold reductions in IKK1- and IKK2-mediated luciferase activity, respectively (FIG. 6A).

Next, we detected IKK activity in MC160-expressing cells via an in vitro kinase assay, in which a reduction in IKK complex formation by the MC160 protein would be measured by the specific associated decreased in phosphorylation of IκBα (FIG. 6B) (38). When the source was lysates from cells over-expressing the IKK1 and IKK2 subunits in the absence of the MC160 protein, a phosphorylated form of recombinant IκBα was easily detected. However, when either MC160 expression also occurred in the cells or if the cells were only transfected with a neutral plasmid, a greatly reduced amount of radioactively-labeled IκBα was observed. Since an external provision of IBα was made to the reactions, the diminution could only be attributed to an inadequate quantity of active IKK complexes either due to intervention by the MC160 protein or to a lack of IKK1 and IKK2 available for complex formation, respectively.

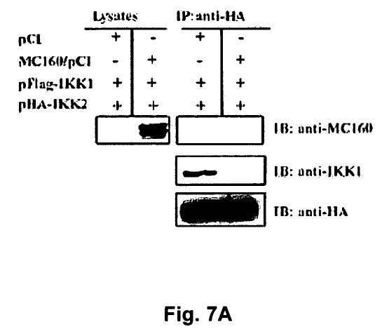

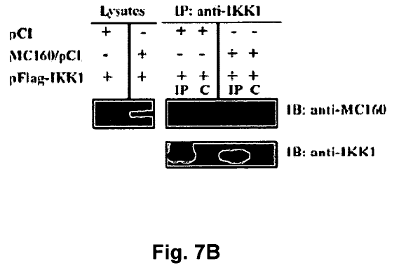

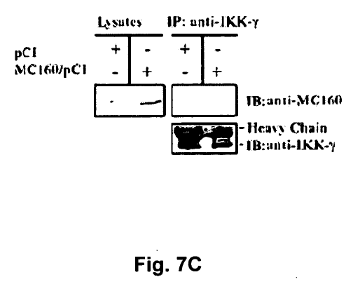

IKK1-IKK2 subunit interactions are undetectable in the presence of the MC160 protein, but MC160 interaction with either protein is undetectable by co-immunoprecipitation. While the IKK heteromeric complex consists of multiple proteins, interactions between the IKK1, IKK2 and IKK-γ proteins are sufficient for IKK activity. If the MC160 protein were to bind to one of these three subunits, then the IKK complex would not be activated. Accordingly, we attempted to detect the association of epitope-tagged IKK1 (FLAG tagged) and IKK2 (HA tagged) molecules in the presence or absence of MC160 by using co-immunoprecipitation assays (FIG. 7A-7C). It appeared that MC160 did not directly interact with the IKK2 protein since MC160 did not co-immunoprecipitate with the HA-tagged IKK2 protein (FIG. 7A). Since similar quantities of HA-tagged IKK2 protein were detected in all immunoprecipitated samples (FIG. 7A), MC160 or IKK1 expression did not appear to impact the plasmid-directed generation of IKK2. Conversely, as expected from the preserved inhibitory ability of the MC160 protein, the increased presence of IKK1 and IKK2 did not prevent production of this viral protein (FIG. 7A). In a similar manner, MC160-IKK1 interactions were undetectable by using co-immunoprecipitation methods (FIG. 7B), although an increased amount of IKK1 was noticed in the immunoprecipitates from samples not expressing the MC160 protein. Interestingly, we observed that IKK1-IKK2 interactions were undetectable when MC160 proteins were expressed (FIG. 7A). This is in contrast to vector-transfected cells, where FLAG-IKK1 proteins were readily found in anti-HA immunoprecipitated samples, demonstrating IKK1-IKK2 interactions (FIG. 7A). The IKK-γ subunit acts as a regulatory protein, stabilizing interactions between IKK1 and IKK2. Since MC160-IKK-γ interactions were not detected via co-immunoprecipitation (FIG. 7C), it seemed unlikely that MC160 directly interacted with this regulatory subunit to prevent IKK1-IKK2 interactions.

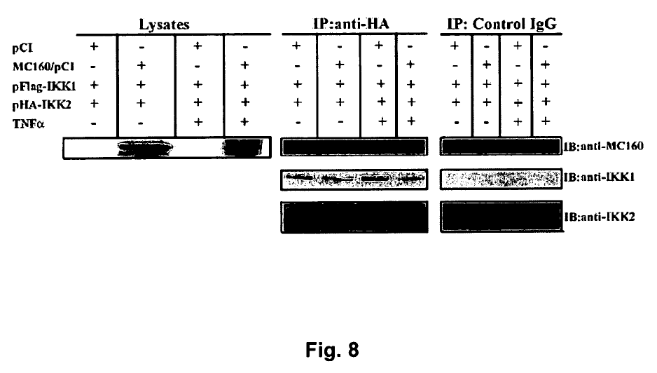

To more closely mimic the physiological signals that an MCV-infected host cell may encounter, we repeated the co-immunoprecipitation experiments, assaying for IKK1-IKK2 interactions in cells incubated with medium containing TNF-α. (FIG. 8). Similar to cells that were incubated in regular medium, decreased levels of IKK1 proteins were present in IKK2 immunoprecipitated samples when MC160 proteins were present (FIG. 8). None of the viral or cellular proteins we were studying non-specifically bound to Protein A-Sepharose beads since neither MC160, IKK1 nor IKK2 was detected in samples that had been immunoprecipitated with a non-specific antibody. Also, MC160 protein levels were equal in lysates from cells that were incubated in medium absent for or containing TNF-α, indicating that the presence of TNF-α did not affect MC160 expression.

MC160 expression coincides with a decrease in IKK1 expression levels. While IKK1 and IKK2 subunits are normally inactive in resting cells, ectopic over-expression of these proteins results in their phosphorylation and subsequent activation, an event detectable by probing immunoblotted lysates from pCI-transfected cells with antisera that simultaneously recognizes phosphorylated IKK1 and IKK2 (FIG. 9). In contrast, there was a decrease in phospho-IKK levels in MC160-expressing cells. This decrease was not due to over-expression of protein since expression of the MCV MC159 product did not significantly alter IKK phosphorylation levels when IKK1 and IKK2 were co-expressed. Further analysis of these lysates revealed that IKK1 levels, but not IKK2 or actin levels, were reduced in MC160-expressing cells as compared to either MC159/pCI- or pCI-transfected cells. Therefore, the possibilities that the transfection process or MC160 expression was globally decreasing protein expression were ruled out. Similar results were observed when only IKK1 was over-expressed: phospho-IKK1 and total IKK1 levels were decreased in MC160-expressing cells as compared to cells transfected with either pCI or MC159/pCI. Phosphorylation of IKK2 was also decreased in cells co-transfected with MC160/pCI and pHA-IKK2, indicating that MC160 inhibited activation of both kinase subunits. Yet, total IKK2 levels were similar in pCI- versus MC160/pCI-transfected cells, indicating that MC160 did not affect IKK2 protein levels. In MC159/pCI-transfected cells, IKK2 was present at levels similar to that seen in pCI-transfected cells, but phospho-IKK2 levels were decreased. Similar results were observed when cells were incubated in medium containing TNF-α, a cytokine known to induce IKK1 and IKK2 phosphorylation.

The MC160, MC160 B, MC160 A+B or MC160 C proteins can each be utilized as a therapeutic agent to control inflammation. Small protein domains from HIV-1 TAT, HSV-1 VP22 and the Antennapedia homeodomain, among others as described herein above, cross cell membranes efficiently and independently of specific receptors. These peptide carriers (termed cell penetration peptide herein) can be noncovalently bound or conjugated (or incorporated within the primary structures through molecular biological technology) with proteins of interest for introducing biologically active therapeutic proteins into cells. For example, MC160 is noncovalently bound to the synthetically-created 21-residue Pep-1 peptide carrier (KETWWETWWTEWSQPKKKRKV, SEQ ID NO:8) (cell penetration peptide) (20). MC160 naturally associates with Pep-1, thus decreasing the possibility that chemical cross-linking alters the biological properties of MC160. (MC160 must be chemically cross-linked or fused by recombinant DNA technology to other peptide carriers.) Pep-1 is neither toxic to cells, nor does it affect the appropriate subcellular localization of the proteins it delivers (20). Pep-1 delivers proteins as large as 119-kDa (20), therefore the 60-kDa MC160 protein is successfully delivered into cells using this particular peptide. To conjugate MC160 to Pep-1, 6X-His-tagged MC160 products (over-expressed in E. coli and purified to homogeneity using nickel and size-exclusion columns) are incubated with purified Pep-1 peptides in PBS. Useful ranges of peptide:MC160 molar ratios are from 1:1 to 1:40.

The delivery of MC160 or its inflammation-inhibiting derivatives can readily be optimized. Each of the MC160-Pep-1 solutions (above) are incubated with 293T cell monolayers. At different times after incubation with MC160-Pep-1 conjugates, intracellular MC160 are detected in fixed cells stained with anti-4×His-FITC antibody, by using confocal microscopy. MC160 stability and cytoplasmic localization over a range of times are also determined by using confocal microscopy. MC160 can also be conjugated to one of the TAT, VP22 or Antennapedia peptides, among others as described herein above, for intracellular delivery.

NF-κB activity and pro-inflammatory cytokine expression after administration of or expression of MC160, MC160B or MC160 C have been studied. After incubation with MC160-Pep-1 complexes, 293T cells were treated with TNF and assayed for NF-κB activation state by EMSAs. As a control, cells incubated with Pep-1 alone or with Pep-1 and MC160 A (a truncated MC160 protein that does not inhibit TNF-induced NF-κB activity, FIG. 4) were assayed as well. Most importantly, the cells were analyzed for proinflammatory molecule expression, either by using reverse transcriptase PCR to measure cytokine gene transcription (see Shisler and Jin for a detailed protocol), or by using ELISAs to detect proinflammatory molecule synthesis.

With exogenously introduced MC160 (or an effective truncated derivative such as MC160 B or MC160 C) inhibiting NF-κB transcriptional function, this protein is useful as a therapeutic. Thus, these assays are repeated with either the DED 2 or C-terminal MC160 regions (regions that possess inhibitory function, FIG. 3B), and the effectiveness of the smaller mutant MC160 molecules is compared to wild type MC160. Further, the ability of exogenously delivered MC160 proteins to inhibit NF-κB activation in human cell lines of different tissue origin, and in response to different extracellular signals (i.e.; LPS, IL-1), is assayed to determine the global function of MC160.

TNF-α is produced by keratinocytes, the natural host cells for MCV (44), in response to stresses including, but not limited to, ultraviolet radiation and endotoxins (41, 42). This powerful proinflammatory cytokine up-regulates the expression of immune molecules via NF-κB activation (43). We have shown that the MC160 protein prevents TNF-α-induced NF-κB activation in 293T cells as a potential mechanism for MCV to inhibit the production of anti-viral immune response molecules. Kidney epithelial-derived 293T cells, while not the natural host cell type for MCV, are a common and well-defined model system for studying TNF-RI-mediated signal transduction, making their use important in characterizing this original function for the MC160 protein. The MC160 protein functions similarly in human keratinocytes, cells that are normally infected by MCV.

The TNF-RI mediates the classical NF-κB activation pathway by virtue of TRADD, RIP and TRAF2 forming a signalsome, that in turn allows IKK to dock to it and become activated (6, 44). We originally hypothesized that MC160 bound to RIP or TRAF2 to prevent IKK from associating with the signalsome and subsequently being phosphorylated, but the lack of detectable interactions between the MC160 protein and either RIP or TRAF2 ruled out this possibility. Expression of the MC160 protein also inhibited NIK- and MyD88-induced NF-κB activation, therefore we believe that MC160 prevents an event common to all of the above pathways, namely IKK activation. This prediction was confirmed; cells expressing the MC160 protein did not possess active, phosphorylated IKK or IKK1-IKK2 complexes necessary for IKK activity.

We did not detect a MC160 protein interaction with IKK1 or IKK2 by co-immunoprecipitation, diminishing the likelihood of that MC160 prevented formation of the IKK complex by binding to either kinase subunit. IKK1-IKK2 interactions, which are necessary for IKK auto-activation, occur via association of both proteins to IKK-γ (40). Since IKK1-IKK2 interactions were undetectable in MC160-expressing cells, another possible mechanism for MC160 function is that the MC160 protein prevents the formation of an IKK complex by binding to the IKK-γ subunit. In this regard, one other viral FLIP that also contains two tandem DEDs, the human herpesvirus-8 (HHV-8) ORF-K13 protein, has previously been shown to interact with IKK-γ albeit to activate NFκB (18). However, MC160 interactions with endogenous IKK-γ were undetectable in our assays, ruling out this putative MC160 mechanism.

We found that phosphorylated IKK1 and IKK2 were dramatically decreased in MC160-expressing cells. Further analysis revealed that total IKK1 levels, but not IKK2 levels, were decreased. Several reports show that the phosphorylation of the IKK2 subunit is essential for TNF-α-induced NF-κB activation, while deletion of the IKK1 subunit does not alter this signal transduction pathway. Without wishing to be bound by theory, we believe that MC160-mediated prevention of IKK2 phosphorylation is the more important mechanism for MC160 function. Other cellular proteins, such as the TANK-binding kinase (TBK1) (45) and ELKS (45) have been shown to interact with and activate IKK subunits as a necessary event during TNF-RI-mediated NF-κB activation. Whether these cellular proteins bind to IKK subunits to enhance IKK stability is not known. Thus, one possibility is that the MC160 protein exerts its effects indirectly by binding to one of these cellular proteins, thereby compromising the stability of the IKK1 protein in activated cells or preventing phosphorylation of either or both subunits. At least one other poxvirus protein, the vaccinia N1L protein, interacts with TBK1 and prevents TNF-α-induced NF-κB activation (33).

Recently, an alternative NF-κB activation pathway, in which IKK1 subunits form a homodimer that phosphorylates IκB proteins to enable NF-κB activation, was characterized (47). In this non-classical cascade, the IKK1 homodimer is phosphorylated by a cellular kinase, such as NIK (48). We have demonstrated that the MC160 protein inhibited both NIK- and IKK1-induced luciferase activities and decreased IKK1 levels; it is possible that MC160 negatively impacts the alternative pathway.

It should be noted that, like the MC160 protein, the MCV MC159 protein contains two tandem death effector domains (DEDs) (24). Interestingly, this virus protein also inhibits TNF-α- and MyD88-induced NF-κB activation (49). However, despite the DED I and DED II regions being 45% and 35% identical at the amino acid level, respectively, between the two virus proteins (24), it appears that they prevent TNF-α-induced NF-κB activation via different mechanisms. For instance, MC160 expression decreases IKK1 levels whereas MC159 does not. Also, the MC160 protein inhibited RIP-induced NF-κB activation (FIG. 5A) while the MC159 protein does not (49), suggesting that only the former affects MEKK-3-induced NF-κB activation (an event mediated by RIP). Moreover, the latter binds to RIP and TRAF2 (5) while such interactions involving the MC160 protein were not detected, indicating that the two proteins target different cellular proteins in vivo. In this regard, the MC160 protein possesses a unique and active C-terminal region.

Recent reports have shown that TNF-α induces a biphasic NF-κB activation response, with the transient phase dependent upon IκBα degradation and the persistent phase reliant on IκBα degradation (53, 54, 55). Not surprisingly, each type of NF-κB activation results in the expression of different chemokines and pro-inflammatory proteins in cultured cells at different times after exposure to TNF-α (52, 53, 54, 55). Because the MC159 protein inhibits IκBα, but not IκBα degradation, it is likely that this viral protein blocks the persistent phase of activation (49). Thus, it is tempting to speculate that MCV expresses the MC160 protein to inhibit the transient NF-κB activation phase, and that both viral proteins are required to completely prevent TNF-α-mediated signaling that host cells may encounter during infection. We have observed that co-expression of the MC160 and MC159 proteins reduces TNF-α-induced luciferase activity to a greater degree than expression of either protein alone.

It appears that blocking NF-κB activation of virus-infected cells is important for poxvirus pathogenesis. In this regard, elimination of the M150R ORF from the myxoma virus genome created a mutant that causes limited disease in rabbits, unlike its extremely virulent progenitor (31). Similarly, mice infected with vaccinia virus whose DNA lacked either the A52R or A46R ORFs, that encode proteins capable of inhibiting Toll-like receptor-induced NF-κB activation, exhibited a decreased virus-induced illness as compared to animals receiving the genetically unaltered parent (50, 51).

The recombinant MC160 proteins and truncated MC160 proteins of the present invention can be readily engineered to facilitate purification and/or immobilization to a solid support of choice. For example, a stretch of 6-8 histidines (“His-tag”) can be engineered through mutagenic polymerase chain reaction, through the use of available cloning vectors or other recombinant DNA technology to allow purification of expressed recombinant protein over a nitrilotriacetic acid (NTA) column using commercially available materials. Oligopeptide “tags” which can be fused to the MC160 protein or a truncated derivative which retains the NF-κB activation-inhibiting activity by such techniques include, without limitation, strep-tag (Sigma-Genosys, The Woodlands, Tex.) which directs binding to streptavidin or its derivative streptactin (Sigma-Genosys); a glutathione-S-transferase gene fusion system which directs binding to glutathione coupled to a solid support (Amersham Pharmacia Biotech, Uppsala, Sweden); a calmodulin-binding peptide fusion system which allows purification using a calmodulin resin (Stratagene, La Jolla, Calif.); a maltose binding protein fusion system allowing binding to an amylose resin (New England Biolabs, Beverly, Mass.); and an oligo-histidine fusion peptide system which allows purification using a Ni²⁺-NTA column (Qiagen, Valencia, Calif.).

All art-known functional equivalents of methods, starting materials, synthetic methods, pharmaceutical formulations and delivery methods are intended to be included in the present invention. Whenever a range is given in the specification, for example, a temperature range, a time range, or a composition range, all intermediate ranges and subranges, as well as all individual values included in the ranges given are specifically included within the scope of the present invention.

As used herein, “comprising” is synonymous with “including,” “containing,” or “characterized by,” and does not exclude additional, unrecited elements or method steps. As used herein, “consisting of” excludes any element, step, or ingredient not specifically recited. As used herein, “consisting essentially of” does not exclude materials or steps that do not materially affect the basic and novel characteristics of the claim. Any recitation herein of the term “comprising”, particularly in a description of components of a composition or in a description of elements of a device, encompasses those compositions and methods consisting essentially of and consisting of the recited components, elements or steps. The invention illustratively described herein suitably may be practiced in the absence of any element or elements, limitation or limitations which is not specifically disclosed herein.

The terms and expressions which have been employed herein are used as terms of description and not of limitation, and there is no intent to exclude any equivalents of the features shown and described or portions thereof, and it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The exact formulation, route of administration and dosage of MCV MC160 or a smaller derivative which inhibits the activation of NF-κB can be chosen by the individual physician in view of the patient's condition (see e.g. Fingl et al., in The Pharmacological Basis of Therapeutics, 1975, Ch. 1 p. 1).

It should be noted that the attending physician knows how to and when to terminate, interrupt, or adjust dose and/or schedule due to toxicity, organ dysfunctions, or successful treatment. Conversely, the attending physician also knows to adjust treatment to higher levels if the clinical response is not adequate (precluding toxicity). The magnitude of an administered dose in the management of the disorder of interest varies with the severity of the condition to be treated and to the route of administration. The severity of the condition may, for example, be evaluated, at least in part, by standard prognostic evaluation methods. Further, the dose and/or dose frequency, also vary according to the age, body weight, and response of the individual patient. Similar considerations apply to veterinary medicine as well.

Depending on the specific conditions being treated and the targeting method selected, a therapeutic composition comprising MC160 or an inflammation-inhibiting derivative thereof as the active ingredient may be formulated and administered systemically or locally, for example, topically. Techniques for formulation and administration are well known to the art. Suitable routes can include, for example, topical, oral, rectal, transdermal, vaginal, transmucosal, or intestinal administration; parenteral delivery, including intramuscular, subcutaneous, or intramedullary injections, as well as intrathecal, intravenous, or intraperitoneal injections or administered into joints.

For injection, the therapeutic agent may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Topical formulations are also well known to the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical particles with aqueous interiors bounded lipid bilayers. Soluble molecules present in an aqueous solution at the time of liposome formation are incorporated into the aqueous interior. The liposomal contents are both protected from the external microenvironment and, because liposomes fuse with cell membranes, the contents are efficiently delivered into the cell cytoplasm. Additionally, due to their hydrophobicity, small organic molecules may be directly administered intracellularly.

Pharmaceutical compositions suitable for use in the present invention include compositions wherein the MC160 protein, MC160 B, MC160 A+B or MC160 C, together with a cell penetration peptide, is present in an effective amount to achieve the intended purpose. Determination of the effective amount is well within the capability of those skilled in the art.

In addition to the active ingredients, these pharmaceutical compositions may contain suitable pharmaceutically acceptable carriers including excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine, as necessary.

The pharmaceutical compositions of the present invention may be manufactured by any means known to the art, including but not limited to, conventional mixing, dissolving, granulating, dragee-making, levitating, emulsifying, encapsulating, entrapping or lyophilizing processes.

Pharmaceutical formulations for parenteral administration include aqueous solutions of the active MC160 protein or inflammation-inhibiting derivative thereof. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. The frequency of dosing depends on the disease treated. Crohn's disease and rheumatoid arthritis, for example, require repeated administrations. For example, for treatment of rheumatoid arthritis, MC160, MC160 B or MC160 C can be administered at one to three month intervals. For a Crohn's disease patient, the initial administration can be followed at two to eight week intervals, as needed. Other chronic inflammatory diseases also require repeated administrations at intervals deemed appropriate by the physician.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Monoclonal or polyclonal antibodies, preferably monoclonal, specifically reacting with a protein or peptide of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

All references cited herein are hereby incorporated by reference to the extent there is no inconsistency with the present disclosure. Patents and publications herein reflect the level of skill of those in the art to which the invention pertains.

Although the description herein contains certain specific descriptions and examples, these should not be construed as limiting the scope of the invention but as merely providing illustrations of some of the presently preferred embodiments of the invention. For example, thus the scope of the invention should be determined by the appended claims and their equivalents, rather than by any examples given.

Plasmids, Viruses, Cells and Antisera

The DNA segment encoding MC160 was cloned into the pCI eukaryotic expression vector (Promega Corporation, Madison, Wis.) under the transcriptional control of the cytomegalovirus promoter (25), and the recombinant plasmid was used for transfection. In some transfection experiments, an HA or polyhistidine epitope tag was engineered by site directed mutagenesis at the N terminus of the MC160 protein. Inflammation-inhibiting derivatives of the MC160 protein can also be expressed in a similar way.

Human embryonic kidney (293T) cells, Jurkat cells and rabbit kidney RK13 cells were obtained from the American Type Culture Collection, Manassas, Va. Adherent HeLa and 293T cells were maintained in DMEM supplemented with 10% fetal bovine serum and glutamine. Alternatively, 293T cells were maintained in Eagle's minimal essential medium supplemented with 10% fetal bovine serum. The H9 suspension T cell line was maintained in RPMI supplemented with 10% fetal bovine serum and glutamine.

Polyclonal rabbit antisera were raised against a peptide sequence unique to MC160 (CTDSEPEDSAGPS, SEQ ID NO:9). Other antibodies were obtained from commercial sources.

The MC160/pCI plasmid contains the intact MC160 open reading frame (ORF) placed under the transcriptional control of a cytomegalovirus promoter (25). The MC159/pCI plasmid was similarly constructed as previously described (25). The parental pCI vector was purchased from Promega. Plasmid pHATRAF2, which produces a hemagglutinin (HA) epitope-tagged TRAF2 protein, was provided by Jonathan Ashwell, National Institutes of Health (35), while plasmid pRIP was a gift from Preet Chaudhary (University of Texas Southwestern Medical Center) (5). Richard Tapping (University of Illinois) provided the pFLAG-MyD88 plasmid, which produces a FLAG epitope-tagged MyD88 protein. The pFLAG-IKK1, pHA-IKK2, and pNIK plasmids were kind gifts from Ulrich Siebenlist (National Institutes of Health) (34). Plasmid pIKK2DN contains an altered IKK2 gene, which expresses a dominant negative, kinase-deficient IKK2 protein (36), was a gift from Mark Hershenson (University of Chicago).

For all experiments, plasmid DNA was introduced into 293T cells using the FuGene 6 transfection reagent (Roche Diagnostics, Basel, CH).

Polyclonal antisera specifically recognizing either the MC160 or MC159 proteins were described previously (25). Anti-HA antiserum was obtained from Sigma-Aldrich. Anti-TRAF2, anti-IKK1, anti-IKK2 (recognizing the unphosphorylated forms), anti-IKK-γ, anti-IκBα, and NF-κB p65 and p50 subunit antibodies were purchased from Santa Cruz Biotechnology, Santa Cruz, Calif. Antisera recognizing the phosphorylated forms of both IKK1 and IKK2 simultaneously were purchased from Cell Signaling Technologies, Danvers, Mass. Anti-RIP antibody was acquired from BD Transduction Laboratories, Madison, Wis. Horseradish peroxidase-conjugated anti-rabbit or anti-mouse immunoglobulin G (IgG) antibodies were obtained from Pierce Biotechnology, Rockford, Ill. Human recombinant TNF-α was purchased from Roche, Basel, CH.

Transfection Assays

293T cells were co-transfected with a 2:1 or 5:1 ratio of pCI-based plasmid to CMVβgal reporter plasmid (Stratagene/Biocrest, Cedar Creek, Tex.) using Fugene 6 (Invitrogen, Carlsbad, Calif.). At 24 hr after transfection, monolayers were incubated with fresh media.

Measurement of NF-κB Activation Via Luciferase Reporter Assays

Subconfluent monolayers of 293T cells in a 12 well plate system were transfected with 100 ng or pRL-null (Promega) and 900 ng pNF-κBluc (Stratagene) and 500 ng plasmid MC160/pCI using the Fugene 6 transfection reagent (Invitrogen). The pRL-null plasmid contains the Renilla reniformis (sea pansy) luciferase gene with no promoter and sea pansy luciferase is constitutively expressed at low levels. The pNF-κBluc plasmid contains the firefly luciferase gene under the transcriptional control of a synthetic promoter containing five direct repeats of the NF-κB binding element. As a result, firefly luciferase expression is induced by activated NF-κB. At 18 to 24 hr post-transfection, cells were treated with media containing TNF (10 ng/ml media). After 4 hr incubation, cell monolayers were lysed in 100 to 250 μl passive lysis buffer (Promega), followed by cell lysates analysis for both luciferase activities by using the dual Luciferase Reporter assay (Promega).

In some experiments, the luciferase reporter assay employed to quantify NF-κB activation was described previously (56). In assays involving TNF-α mediated activation, subconfluent 293T cell monolayers were co-transfected with pNF-κBluc (450 ng), pRL-null (50 ng) and 500 ng of either pCI or MC160/pCI, unless otherwise stated. At 24 h post-transfection, monolayers were overlaid with fresh medium lacking or containing TNF-α (10 ng/ml, unless otherwise stated). At varying times afterwards, cell monolayers were lysed in passive lysis buffer (Promega) and the lysates were evaluated for sea pansy and firefly luciferase activities by using the Dual Luciferase Reporter Assay (Promega).

In other luciferase assays, NF-κB was first activated due to over-expression of either the RIP, TRAF2, MyD88, NIK, IKK or IKK2 signaling proteins. In this case, subconfluent 293T cells were co-transfected with pNF-κBluc (450 ng), pRL-null (50 ng), and either pCI (500 ng) or MC160/pCI (500 ng) and either pHA-TRAF2 (500-2000 ng), pHA-RIP (50-500 ng), pFLAG-MyD88 (250 ng), pNIK (10-50 ng), pFLAG-IKK1 (500 ng) or pHA-IKK2 (500 ng). When required, greater amounts of pCI were included in the transfection mixture to equalize the quantity of DNA present. At 24 h post-transfection, cell monolayers were lysed and assayed as described above.

Luciferase activity was measured as relative light units (RLUs) by using Luminoskan Microplate Luminometer (Thermo Electron Corporation, Waltham, Mass.). For all assays, experiments were performed in triplicate. For each experimental point, the average of the firefly luciferase activity was divided by the average of the sea pansy luciferase activity too correct for differences in transfection efficiencies. The resulting ratios were used to compare the expression of the firefly luciferase gene in cells containing and expressing the MC160 protein or a fragment of the MC160 protein to that present in cells lacking the MC160 protein or fragment thereof.

Some of the lysates generated during the luciferase assays were also analyzed for the presence of either MC160, RIP, TRAF2, IKK1 or IKK2 by immunoblotting. The protein concentration of each lysate was determined using a bicinchoninic acid (BCA) assay (Pierce). Ten μg of protein was separated electrophoretically in a 12% SDS polyacrylamide gel and transferred to a PVDF membrane (Millipore). Membranes were first incubated with the indicated primary antibodies and then incubated with a secondary antibody conjugated to horseradish peroxidase. Blots were developed using Pierce Super Signal West chemiluminescence reagents per manufacturer's recommendations.

Detection of NF-κB-Regulated Host TNF Gene Transcription Using RT-PCR

293T cells were transfected with plasmids expressing the MC160 protein and fragments thereof. At 24 hr post transfection, cells were treated with TNF for varying times, collected, total RNA was extracted from those cells using the Qiagen RNeasy kit (Qiagen, Valencia, Calif.) and 3 μg in a 50 μl reaction mixture was reverse transcribed into single-stranded cDNA with Superscript II reverse transcriptase (Invitrogen) and oligo(dT) primers. For all PCRs, 1 μl of template cDNA was used. Amplification of tumor necrosis factor (TNF) and glyceraldehydes 3-phosphate dehydrogenase (GAPDH) cDNA were performed in parallel by using PCR with primers specific for human (293T) sequences. Primers for human TNF were 5′-GAGTGACAAGCCTGTAGCCCATGTTGTAGCA-3′ (forward, SEQ ID NO:10) and 5′-GGCAATGATGATCCCAAAGTAGACCTGCCCAGACT-3′, (reverse, SEQ ID NO:11) to yield a 480 bp product. PCR conditions were 95° C. for 45 s, 60° C. for 45 s, and 72° C. for 2 min. Primers for human GAPDH were 5′-TGAAGGTCGGAGTCAACGGATTTGGT-3′ (forward, SEQ ID NO:12) and 5′-CATGTGGGCCATGAGGTCCACCAC-3′ (reverse, SEQ ID NO:13) to yield a 921 bp product. PCR conditions were 95° C. for 30 s, 61° C. for 1 min and 72° C. for 1 min. TNF and GAPDH cDNAs were amplified for either 35 cycles (rabbit) or 25 cycles (human). A portion of each PCR was analyzed by agarose gel electrophoresis and amplicons were visualized by ethidium bromide staining.

Electromobility Shift Assays

Subconfluent monolayers of 293T cells were transfected with 1 μg of either pCI or MC160/pCI. At 24 h post-transfection, cells were incubated with medium lacking or containing TNF-α (10 ng/ml) for the times indicated. Afterwards, cells were collected at various times and lysed in CE buffer (37) to disrupt the cytoplasmic, but not nuclear, membranes. Nuclei were separated from lysates by centrifugation and washed in excess CE buffer to remove contaminating cytoplasmic proteins. Next, nuclear membranes were disrupted in the presence of NE buffer (37). Two μg of protein from each nuclear extract were incubated with 0.35 pmol of [α-32P] labeled, double-stranded oligonucleotides containing binding sites for the NF-κB transcription factor (5′-AGTTGAGGGGACTTTCCCAGGC-3′; SEQ ID NO:14) (Promega) in gel shift binding buffer (Promega) for 20 min at room temperature. Each reaction product was analyzed electrophoretically in a 6% polyacrylamide gel (Invitrogen) under non-denaturing conditions. Dried gels were exposed to phosphorimager plates (Molecular Devices) and images were developed and analyzed using the ImageGauge and ImageReader programs (Fuji). For some reactions, an excess of non-radiolabeled oligonucleotides (1.75 pmol) (Promega) possessing or lacking the NF-κB recognition site or 2 μg of monoclonal antibodies recognizing either the NF-κB p65 or p50 subunits (Santa Cruz Biotechnology) were included.

Co-Immunoprecipitation assays.

Co-immunoprecipitations were performed as described previously (25). Briefly, subconfluent monolayers of 293T cells were co-transfected with equal amounts of the indicated plasmids. At 24 h post-transfection, some cells were incubated in medium containing TNF-α (10 ng/ml) for 15 min. Next, cells were harvested by scraping a n d pelleted by centrifugation at 10,000×g for 10 seconds. Cells were then lysed in DED-co-immunoprecipitation lysis buffer (46) during a for 30 min incubation at 4° C. and then centrifuged at 10,000×g and 4° C. for 10 min. Supernatants were removed and incubated with either anti-HA Affinity Matrix beads (Roche) or with antibodies and protein A-Sepharose beads (Amersham) for 1 h at 4° C., with constant rotation. Beads and bound proteins were pelleted by a brief high-speed centrifugation (10,000×g for 30 s) and washed several times in lysis buffer. Pelleted proteins were resuspended in Laemmli buffer (Pierce), boiled for 5 min and analyzed by SDS-PAGE. Proteins were electrophoretically transferred to a PVDF membrane and subsequently the blots were analyzed by immunoblotting with specific primary antibodies and a horseradish-conjugated secondary antibody. Blots were developed as described above. Pre-immunoprecipitated lysates were analyzed for protein expression in a similar manner.

In Vitro Kinase Assays.

In vitro kinase assays were performed as described before (38). Briefly, subconfluent monolayers of 293T cells were co-transfected with pFLAG-IKK1 (250 ng), pHA-IKK2 (250 ng) and either MC160/pCI (1500 ng) or pCI (1500 ng). At 24 h post-transfection, cells were harvested, collected by low-speed centrifugation, and lysed in kinase assay lysis buffer (38) for 10 min at 4° C. and then centrifuged at 10,000×g for 10 min. Supernatants were incubated with anti-IKK1 antibody and protein A-Sepharose beads for 1 h at 4° C. with continuous rocking. Beads were collected by centrifugation, washed 3 times with lysis buffer, once with kinase assay kinase buffer (38), and then incubated in kinase buffer containing 5 μM ATP, 5 μCi [α-32P] ATP and 1 μg of recombinant IκBα (Cell Sciences, Canton, Mass.) for 20 min at 30° C. After addition of 5× Laemmli buffer, each sample was boiled for 5 min. Each reaction was separated by 12% SDS-PAGE and the proteins were electrophoretically transferred to a PVDF membrane. Blots were exposed to a phosphorimaging plate (Molecular Devices, Sunnyvale, Calif.). After an overnight incubation, the plates were analyzed by using the ImageQuant and ImageReader software (Fuji). The immunoblots were probed first with anti-IκBα antibodies, and then with anti-rabbit IgG antibodies conjugated to alkaline phosphatase. Antigen-antibody complexes were detected performed by using an alkaline phosphate substrate according to manufacturer's directions (Promega).

### BIBLIOGRAPHY

1. Baldi, L., K. Brown, G. Franzoso, and U. Siebenlist. 1996. Critical role for lysines 21 and 22 in signal-induced, ubiquitin-mediated proteolysis of I kappa B-alpha. J. Biol. Chem. 271:376-379.
2. Bhawan, J., Y. Dayal, and A. K. Bhan. 1986. Langerhans cells in molluscum contagiosum, verruca vulgaris, plantar wart, and condyloma acuminatum. J. Am. Acad. Dermatol. 15:645-649.
3. Brockman, J. A., D. C. Scherer, T. A. McKinsey, S. M. Hall, X. Qi, W. Y. Lee, and D. W. Ballard. 1995. Coupling of a signal response domain in I kappa B alpha to multiple pathways for NF-kappa B activation. Mol. Cell. Biol. 15:2809-2818.
4. Brown, K., S. Gerstberger, L. Carlson, G. Franzoso, and U. Siebenlist. 1995. Control of I kappa B-alpha proteolysis by site-specific, signal-induced phosphorylation. Science 267:1485-1488.
5. Chaudhary, P. M., A. Jasmin, M. T. Eby, and L. Hood. 1999. Modulation of the NF-kappa B pathway by virally encoded death effector domains-containing proteins. Oncogene 18:5738-5746.
6. Chen, G., and D. V. Goeddel. 2002. TNF-R1 signaling: a beautiful pathway. Science 296:1634-1635.
7. Chen, Z., J. Hagler, V. J. Palombella, F. Melandri, D. Scherer, D. Ballard, and T. Maniatis. 1995. Signal-induced site-specific phosphorylation targets I kappa B alpha to the ubiquitin-proteasome pathway. Genes Dev. 9:1586-1597.
8. DiDonato, J., F. Mercurio, C. Rosette, J. Wu-Li, H. Suyang, S. Ghosh, and M. Karin. 1996. Mapping of the inducible IkappaB phosphorylation sites that signal its ubiquitination and degradation. Mol. Cell. Biol. 16:1295-1304.
9. DiDonato, J. A., M. Hayakawa, D. M. Rothwarf, E. Zandi, and M. Karin. 1997. A cytokine-responsive IkappaB kinase that activates the transcription factor NF-kappaB. Nature 388:548-554.
10. Garvey, T., J. Bertin, R. Siegel, M. Lenardo, and J. Cohen. 2002. The death effector domains (DEDs) of the molluscum contagiosum virus MC159 v-FLIP protein are not functionally interchangeable with each other or with the DEDs of caspase-8. Virology 300:217-225.
11. Ghosh, S., M. J. May, and E. B. Kopp. 1998. NF-kappa B and Rel proteins: evolutionarily conserved mediators of immune responses. Annu. Rev. Immunol. 16:225-260.
12. Gottlieb, S. L., and P. L. Myskowski. 1994. Molluscum contagiosum. Int. J. Dermatol. 33:453-461.
13. Heng, M. C., M. E. Steuer, A. Levy, S. McMahon, M. Richman, S. G. Allen, and B. Blackhart. 1989. Lack of host cellular immune response in eruptive molluscum contagiosum. Am. J. Dermatopathol. 11:248-254.
14. Karin, M., and B. Ben-Neriah. 2000. Phosphorylation meets ubiquitination: the control of NF-kB activity. Annu. Rev. Immunol. 18:621-663.
15. Kataoka, T., R. C. Budd, N. Holler, M. Thome, F. Martinon, M. Irmler, K. Burns, M. Hahne, N. Kennedy, M. Kovacsovics, and J. Tschopp. 2000. The caspase-8 inhibitor FLIP promotes activation of NF-kappaB and Erk signaling pathways. Curr. Biol. 10:640-648.
16. Konya, J., and C. H. Thompson. 1999. Molluscum contagiosum virus: antibody responses in persons with clinical lesions and seroepidemiology in a representative Australian population. J. Infect. Dis. 179:701-704.
17. Legler, D. F., O. Micheau, M. A. Doucey, J. Tschopp, and C. Bron. 2003. Recruitment of TNF receptor 1 to lipid rafts is essential for TNFalpha-mediated NF-kappaB activation. Immunity 18:655-664.
18. Liu, L., M. T. Eby, N. Rathore, S. K. Sinha, A. Kumar, and P. M. Chaudhary. 2002. The human herpes virus 8-encoded viral FLICE inhibitory protein physically associates with and persistently activates the Ikappa B kinase complex. J. Biol. Chem. 277:13745-13751.
19. Mercurio, F., H. Zhu, B. W. Murray, A. Shevchenko, B. L. Bennett, J. Li, D. B. Young, M. Barbosa, M. Mann, A. Manning, and A. Rao. 1997. IKK-1 and IKK-2: cytokine-activated IkappaB kinases essential for NF-kappaB activation. Science 278:860-866.
20. Morris, M. C., J. Depollier, J. Mery, F. Heitz, and G. Divita. 2001. A peptide carrier for the delivery of biologically active proteins into mammalian cells. Nat. .Biotechnol. 19:1173-1176.
21. Moss, B., J. L. Shisler, Y. Xiang, and T. Senkevich. 2000. Immune-defense molecules of Molluscum contagiosum virus: a human poxvirus. Trends in Microbiology 8:473-477.
22. Scherer, D. C., J. A. Brockman, Z. Chen, T. Maniatis, and D. W. Ballard. 1995. Signal-induced degradation of I kappa B alpha requires site-specific ubiquitination. Proc. Natl. Acad. Sci. USA 92:11259-11263.
23. Senkevich, T. G., J. J. Bugert, J. R. Sisler, E. V. Koonin, G. Darai, and B. Moss. 1996. Genome sequence of a human tumorigenic poxvirus: prediction of specific host response-evasion genes. Science 273:813-816.
24. Senkevich, T. G., E. V. Koonin, J. J. Bugert, G. Darai, and B. Moss. 1997. The genome of molluscum contagiosum virus: analysis and comparison with other poxviruses. Virology 233:19-42.
25. Shisler, J. L., and B. Moss. 2001. Molluscum contagiosum virus inhibitors of apoptosis: The MC159 v-FLIP protein blocks Fas-induced activation of procaspases and degradation of the related MC160 protein. Virology 282:14-25.
26. Traenckner, E. B., H. L. Pahl, T. Henkel, K. N. Schmidt, S. Wilk, and P. A. Baeuerle. 1995. Phosphorylation of human I kappa B-alpha on serines 32 and 36 controls I kappa B-alpha proteolysis and NF-kappa B activation in response to diverse stimuli. EMBO J. 14:2876-2883.
27. Viac, J., and Y. Chardonnet. 1990. Immunocompetent cells and epithelial cell modifications in molluscum contagiosum. J. Cutan. Pathol. 17:202-205.
28. Watanabe, T., K. Nakamura, M. Wakugawa, A. Kato, Y. Nagai, T. Shioda, A. Iwamoto, and K. Tamaki. 2000. Antibodies to molluscum contagiosum virus in the general population and susceptible patients. Arch. Dermatol. 136:1518-1522.
29. Shisler, J. and B. Moss. 2001. Molluscum contagiosum virus inhibitors of apoptosis: The MC159 v-FLIP protein blocks Fas-induced activation of procaspases and degradation of the related MC160 protein. Virology 282:14-25.
30. Hiscott, J., H. Kwon, and P. Genin. 2001. Hostile takeovers: viral appropriation of the NF-kappaB pathway. J. Clin. Invest. 107:143-151.
31. Camus-Bouclainville, C., L. Fiette, S. Bouchiha, B. Pignolet, D. Counor, C. Filipe, J. Gelfi, and F. Messud-Petit. 2004. A virulence factor of myxoma virus colocalizes with NF-kappaB in the nucleus and interferes with inflammation. J. Virol. 78:2510-2516.
32. Bowie, A., E. Kiss-Toth, J. A. Symons, G. L. Smith, S. K. Dower, and L. A. O'Neill. 2000. A46R and A52R from vaccinia virus are antagonists of host IL-1 and toll-like receptor signaling. Proc. Natl. Acad. Sci. USA 97:10162-10167.
33. DiPerna, G., J. Stack, A. G. Bowie, A. Boyd, G. Kotwal, Z. Zhang, S. Arvikar, E. Latz, K. A. Fitzgerald, and W. L. Marshall. 2004. Poxvirus protein N1L targets the 1-kappaB kinase complex, inhibits signaling to NF-kappaB by the tumor necrosis factor superfamily of receptors, and inhibits NF-kappaB and IRF3 signaling by toll-like receptors. J. Biol. Chem. 279:36570-36578.
34. Muller, J. R., and U. Siebenlist. 2003. Lymphotoxin beta receptor induces sequential activation of distinct NF-kappa B factors via separate signaling pathways. J. Biol. Chem. 278:12006-12012.
35. Li, X., Y. Yang, and J. D. Ashwell. 2002. TNF-RII and c-IAP1 mediate ubiquitination and degradation of TRAF2. Nature 416:345-347.
36. Zhou, A., S. Scoggin, R. B. Gaynor, and N. S. Williams. 2003. Identification of NF-kappa B-regulated genes induced by TNFalpha utilizing expression profiling and RNA interference. Oncogene 22:2054-2064.
37. Oie, K. L., and D. J. Pickup. 2001. Cowpox virus and other members of the orthopoxvirus genus interfere with the regulation of NF-kappaB activation. Virology 288:175-187.
38. Fiorentino, L., C. Stehlik, V. Oliveira, M. E. Ariza, A. Godzik, and J. C. Reed. 2002. A novel PAAD-containing protein that modulates NF-kappa B induction by cytokines tumor necrosis factor-alpha and interleukin-1 beta. J. Biol. Chem. 277:35333-35340.
39. Hsu, H., H. B. Shu, M. G. Pan, and D. V. Goeddel. 1996. TRADD-TRAF2 and TRADD-FADD interactions define two distinct TNF receptor 1 signal transduction pathways. Cell 84:299-308.
40. Hayden, M. S., and S. Ghosh. 2004. Signaling to NF-kappaB. Genes Dev. 18:2195-2224.
41. Kock, A., T. Schwarz, R. Kirnbauer, A. Urbanski, P. Perry, J. C. Ansel, and T. A. Luger. 1990. Human keratinocytes are a source for tumor necrosis factor alpha: evidence for synthesis and release upon stimulation with endotoxin or ultraviolet light. J. Exp. Med. 172:1609-1614.
42. Lisby, S., K. M. Mullser, C. V. Jongeneel, J. H. Saurat, and C. Hauser. 1995. Nickel and skin irritants up-regulate tumor necrosis factor-alpha mRNA in keratinocytes by different but potentially synergistic mechanism. Int. Immunology 7:343-352.
43. Esposito, J., and F. FF. 2001. Poxviruses, p. 2885-2922. In K. DM and H. PM (ed.), Fields Virology, vol. 2. Lippincott Williams & Wilkins, Philadelphia.
44. Devin, A., A. Cook, Y. Lin, Y. Rodriguez, M. Kelliher, and Z. Liu. 2000. The distinct roles of TRAF2 and RIP in IKK activation by TNF-R1: TRAF2 recruits IKK to TNF-R1 while RIP mediates IKK activation. Immunity 12:419-429.
45. Pomerantz, J. L., and D. Baltimore. 1999. NF-kappaB activation by a signaling complex containing TRAF2, TANK and TBK1, a novel IKK-related kinase. EMBO J. 18:6694-6704.
46. Ducut Sigala, J. L., V. Bottero, D. B. Young, A. Shevchenko, and F. Mercurio. 2004. Activation of transcription factor NF-kappaB requires ELKS, an IkappaB kinase regulatory subunit. Science 304:1963-1967.
47. Bonizzi, G., and M. Karin. 2004. The two NF-kappaB activation pathways and their role in innate and adaptive immunity. Trends Immunol. 25:280-288.
48. Nakano, H., M. Shindo, S. Sakon, S. Nishinaka, M. Mihara, H. Yagita, and K. Okumura. 1998. Differential regulation of IkappaB kinase alpha and beta by two upstream kinases, NF-kappaB-inducing kinase and mitogen-activated protein kinase/ERK kinase kinase-1. Proc. Natl. Acad. Sci. USA 95:3537-3542.
49. Murao, L., and J. Shisler. 2005. The MCV MC159 protein inhibits late, but not early, events of TNF-alpha induced NF-kappaB activation. Virology 340:255-264.
50. Harte, M. T., I. R. Haga, G. Maloney, P. Gray, P. C. Reading, N. W. Bartlett, G. L. Smith, A. Bowie, and L. A. O'Neill. 2003. The poxvirus protein A52R targets Toll-like receptor signaling complexes to suppress host defense. J. Exp. Med. 197:343-351.
51. Fitzgerald, G. L. Smith, and A. G. Bowie. 2005. Vaccinia virus protein A46R targets multiple Toll-like-interleukin-1 receptor adaptors and contributes to virulence. J. Exp. Med. 201:1007-1018.
52. Hoffmann, A., T. H. Leung, and D. Baltimore. 2003. Genetic analysis of NF-kappaB/Rel transcription factors defines functional specificities. EMBO J. 22:5530-5539.
53. Hoffmann, A., A. Levchenko, M. L. Scott, and D. Baltimore. 2002. The IkappaB-NF-kappaB signaling module: temporal control and selective gene activation. Science 298:1241-1245.
54. Schmidt, C., B. Peng, Z. Li, G. M. Sclabas, S. Fujioka, J. Niu, M. Schmidt-Supprian, D. B. Evans, J. L. Abbruzzese, and P. J. Chiao. 2003. Mechanisms of proinflammatory cytokine-induced biphasic NF-kappaB activation. Mol. Cell 12:1287-1300.
55. Thompson, J. E., R. J. Phillips, H. Erdjument-Bromage, P. Tempst, and S. Ghosh. 1995. I kappa B-beta regulates the persistent response in a biphasic activation of NF-kappa B. Cell 80:573-582.
56. Shisler, J. L., and X. L. Jin. 2004. The vaccinia virus K1L gene product inhibits host NF-kappaB activation by preventing IkappaBalpha degradation. J. Virol. 78:3553-3560.
57. Ling, L., Z. Cao, and D. V. Goeddel. 1998. NF-kappaB-inducing kinase activates IKK-alpha by phosphorylation of Ser-176. Proc. Natl. Acad. Sci. USA 95:3792-3797.

58. Medzhitov, R., P. Preston-Hurlburt, E. Kopp, A. Stadlen, C. Chen, S. Ghosh, and C. A. Janeway, Jr. 1998. MyD88 is an adaptor protein in the hToll/IL-1 receptor family signaling pathways. Mol. Cell 2:253-258.

TABLE 1

AMINO ACID SEQUENCE OF THE MOLLUSCUM CONTAGIOSUM
VIRUS MC160 PROTEIN (SEQ ID NO:1)

MAHEPIPFSF LRNLLAELDA SEHEVLRFLC RDVAPASKTA
EDALRALQRR RLLTLSSMAE LLCALRRFDV LKVRFGMTRE
CAGRLLGHGF LSQYRLQVAA INNMVGSEDL RVMCLCAGKL
LPPSCTPRCL VDLVSALEDA GAISPQDVSV LVTLLHAVCR
YDLSVALSAV AHGHMTVGVG TPVQDEPMDV LEVDDAEPME
ATPACDEIGV VKLAGAASAG APLADGAFAA CTSAGKGEDL
ATSDLTDSEP EDSVFAVADP VYADVDLSMF VRANATADSS
MFVNADAGAD SSLVNADAGA DSSLVNADAG ADSSLVNAVA
DANSSLMRTT SACTDSEPED SAGPSCAGMA LSMFGRAKSV
SSLLLRTKAS Y

We claim:

1. A composition comprising a Molluscum Contagiosum Virus MC160 protein or an inflammation-inhibiting polypeptide derived in amino acid sequence therefrom and a cell penetration peptide, and optionally further comprising a pharmaceutically acceptable carrier

2. The composition of claim 1 wherein the MC160 protein has the amino acid sequence given in SEQ ID NO:1.

3. The composition of claim 1 wherein the inflammation-inhibiting polypeptide has an amino acid sequence selected from the group consisting of amino acids 1 to 170, 82-371,169-371 and 82-220 of SEQ ID NO:1.

4. The composition of claim 1 wherein the cell penetration peptide is derived in amino acid sequence from the Human Immunodeficiency Virus Tat1 protein, from a Drosophila Antennapedia protein, the human Clock protein, hPer1, hPer2, an arginine-rich peptide of from 10 to 50 amino acids in length, or a peptide characterized by the amino acid sequence KETWWETWWTEWSQPKKKRKV (SEQ ID NO:8).

5. The composition of claim 4, wherein the Tat1-derived peptide comprises the amino acid sequence YGRKKRRQRRR (SEQ ID NO:2) or the amino acid sequence RKKRRQRRRAHQ (SEQ ID NO:3).

6. The composition of claim 4, wherein the Antennapedia-derived peptide comprises the amino acid sequence RQRIKIWFQNRRMKWKK (SEQ ID NO:4).

7. The composition of claim 4, wherein the Clock-derived peptide comprises the amino acid sequence RVSRNKSEKKRR (SEQ ID NO:5).

8. A method for decreasing inflammation, said method comprising the step of administering to an area of a human or animal in need of a decrease in inflammation, an effective dose of the composition of claim 1.

9. The method of claim 8, wherein said dose is applied to skin of a human or animal at a site in need of a decrease in inflammation.

10. The method of claim 8, wherein an effective dose of the composition is applied to a joint in need of a decrease in inflammation.

11. The method of claim 10, wherein the joint is affected by rheumatoid arthritis

12. The method of claim 8, wherein said dose is administered intravenously.

13. The method of claim 12, wherein the human or animal suffers from rheumatoid arthritis, Crohn's disease, juvenile rheumatoid arthritis, psoriatic arthritis, ankylosing spondylitis, uveitis, psoriasis or sarcoidosis.

14. The method of claim 13, wherein the intestinal tissue is affected by Crohn's disease.

15. The method of claim 14, wherein an effective dose of the composition is applied to intestinal tissue in need of a decrease in inflammation.

16. The method of claim 14, wherein an effective dose of the composition is administered intravenously.


SEQUENCE LISTING (14 sequences)

SEQ ID NO: 1 - protein, 371 aa, Molluscum contagiosum virus
MAHEPIPFSFLRNLLAELDASEHEVLRFLCRDVAPASKTAEDALRALQRRRLLTLSSMAELLCALRRFDVLKVRFGMTRECAGRLLGHGFLSQYRLQVAAINNMVGSEDLRVMCLCAGKLLPPSCTPRCLVDLVSALEDAGAISPQDVSVLVTLLHAVCRYDLSVALSAVAHGHMTVGVGTPVQDEPMDVLEVDDAEPMEATPACDEIGVVKLAGAASAGAPLADGAFAACTSAGKGEDLATSDLTDSEPEDSVFAVADPVYADVDLSMFVRANATADSSMFVNADAGADSSLVNADAGADSSLVNADAGADSSLVNAVADANSSLMRTTSACTDSEPEDSAGPSCAGMALSMFGRAKSVSSLLLRTKASY

SEQ ID NO: 2 - protein, 11 aa, Artificial
  cell penetration peptide derived from HIV Tat protein
YGRKKRRQRRR

SEQ ID NO: 3 - protein, 12 aa, Artificial
  cell penetration peptide derived from HIV Tat protein
RKKRRQRRRAHQ

SEQ ID NO: 4 - protein, 17 aa, Artificial
  cell penetration peptide derived from Drosophila Antennapedia protein
RQRIKIWFQNRRMKWKK

SEQ ID NO: 5 - protein, 12 aa, Artificial
  cell penetration peptide derived from human Clock protein
RVSRNKSEKKRC

SEQ ID NO: 6 - protein, 15 aa, Artificial
  cell penetration peptide derived from hPer1 protein
RRHHCRSKAKRSRHH

SEQ ID NO: 7 - protein, 18 aa, Artificial
  cell penetration peptide derived from hPer2 protein
KKTGKNRKLKSKRVKPRD

SEQ ID NO: 8 - protein, 21 aa, Artificial
  artificial cell penetration peptide
KETWWETWWTEWSQPKKKRKV

SEQ ID NO: 9 - protein, 13 aa, Artificial
  antigenic oligopeptide from MCV MC160 protein
CTDSEPEDSAGPS

SEQ ID NO: 10 - DNA, 31 bp, Artificial
  Oligonucleotide useful as primer for human Tumor Necrosis Factor
gagtgacaagcctgtagcccatgttgtagca

SEQ ID NO: 11 - DNA, 35 bp, Artificial
  Oligonucleotide useful as primer for human Tumor Necrosis Factor
ggcaatgatgatcccaaagtagacctgcccagact

SEQ ID NO: 12 - DNA, 26 bp, Artificial
  Oligonucleotide useful as primer for human GAPDH
tgaaggtcggagtcaacggatttggt

SEQ ID NO: 13 - DNA, 24 bp, Artificial
  Oligonucleotide useful as primer for human GAPDH
catgtgggccatgaggtccaccac

SEQ ID NO: 14 - DNA, 22 bp, Artificial
  Oligonucleotide containing binding site(s) for NFkappaB transcription factor
agttgaggggactttcccaggc